(19) United States
(12) Patent Application Publication
(10) Pub. No.: US 2011/0053770 A1
(43) Pub. Date: Mar. 3, 2011

(54) SUBSTITUTED AMINOTHIAZOLES AND THEIR USE AS FUNGICIDES

(75) Inventors: Kerstin ILG, Koln (DE); Thomas Seitz, Langenfeld (DE); Klaus Kunz, Dusseldorf (DE); Reiner Fischer, Monheim (DE); Peter Dahmen, Neuss (DE); Arnd Voerste, Koln (DE); Ulrike Wachendorff-Neumann, Neuwied (DE); Jürgen Benting, Leichlingen (DE); Benoit Hartmann, Ste Foy Les Lyon (FR)

(73) Assignee: BAYER CROPSCIENCE AG, MONHEIM (DE)

(21) Appl. No.: 12/836,816

(22) Filed: Jul. 15, 2010

Related U.S. Application Data
(60) Provisional application No. 61/226,402, filed on Jul. 17, 2009.

(30) Foreign Application Priority Data
Jul. 17, 2009 (EP) 09165775.9

(51) Int. Cl. A01C 1/06; C07D 401/12; A01N 43/40; C07D 401/14; C07D 417/12; A01N 43/78; A01P 1/00

(52) U.S. Cl. 504/100; 546/209; 514/326; 546/193; 514/318; 548/193; 514/370

(57) ABSTRACT

The present invention relates to aminothiazoles of the general formula (I), to a process for their preparation, to the use of the aminothiazoles according to the invention for controlling unwanted microorganisms and to a composition for this purpose which comprises the aminothiazoles according to the invention. The invention furthermore relates to a method for controlling unwanted microorganisms by applying the aminothiazoles according to the invention to the microorganisms and/or their habitat.

### CROSS REFERENCE TO RELATED APPLICATIONS

This application claims priority to EP 09165775.9 filed Jul. 17, 2009 and U.S. Provisional Application Ser. No. 61/226,402 filed Jul. 17, 2009, the contents of which are incorporated herein by reference in their entireties.

### BACKGROUND

1. Field of the Invention

The present invention relates to aminothiazoles of the general formula (I), to a process for their preparation, to the use of the aminothiazoles according to the invention for controlling unwanted microorganisms and to a composition for this purpose which comprises the aminothiazoles according to the invention. The invention furthermore relates to a method for controlling unwanted microorganisms by applying the aminothiazoles according to the invention to the microorganisms and/or their habitat.

2. Description of Related Art

WO-A-00/046 184 discloses the use of amidines as fungicides.

WO-A-03/093 224 discloses the use of arylamidine derivatives as fungicides.

WO-A-03/024 219 discloses fungicide compositions comprising at least one N2-phenylamidine derivative in combination with a further selected known active compound.

WO-A-04/037 239 discloses fungicidal medicaments based on N2-phenylamidine derivatives.

WO-A-07/031,513 discloses thiadiazolyl-substituted phenylamidines and their preparation and use as fungicides.

The activity of the amidines described in the prior art is good; however, in some cases it is unsatisfactory.

### SUMMARY

Accordingly, it is the object of the present invention to provide compounds having an improved fungicidal activity.

Surprisingly, this object was achieved by aminothiazoles of the formula (I)

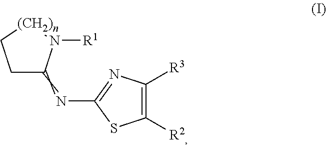

in which
R¹ is selected from the group consisting of hydrogen; straight-chain or branched C₁₋₁₂-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, —COOR′, —COR′, —CONR₂′, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN and —CONR₂′, where R′ is hydrogen or a straight-chain or branched C₁₋₁₂-alkyl, C₁₋₆-haloalkyl or cyclic C₃₋₈-alkyl group which may have 1 to 6 halogen atoms;
R² is selected from the group consisting of hydrogen and straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN and —CONR₂′, where R′ has the meanings given above;
R³ is selected from the group consisting of straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN, C₅₋₁₈-aryl, C₅₋₁₈-aryloxy, C₂₋₁₂-alkenyloxy, C₇₋₁₉-aralkyl and —CONR₂′, where R′ has the meanings given above and the C₅₋₁₈-aryl, C₅₋₁₈-aryloxy, C₂₋₁₂-alkenyloxy and C₇₋₁₉-aralkyl groups may be substituted by one, two or more radicals selected from the group consisting of —R′, —X, —OR′, —SR′, —NR′₂, —SiR′₃, —COOR′, —CN, aryloxy and —CONR₂′, where R′ has the meanings given above;

n is 0, 1 or 2;

and their salts, N-oxides, metal complexes and stereoisomers.

### DETAILED DESCRIPTION OF A PREFERRED EMBODIMENT

### General Definitions

In the context of the present invention, the term halogens (X) comprises, unless defined otherwise, elements selected from the group consisting of fluorine, chlorine, bromine and iodine, with fluorine, chlorine and bromine being used preferably and fluorine and chlorine being used particularly preferably.

Optionally substituted groups can be mono- or polysubstituted, where in the case of polysubstitutions the substituents can be identical or different.

Alkyl groups substituted by one or more halogen atoms (—X) are, for example, selected from the group consisting of trifluoromethyl (CF₃), difluoromethyl (CHF₂), CF₃CH₂, ClCH₂, CF₃CCl₂.

In the context of the present invention, alkyl groups are, unless defined otherwise, straight-chain, branched or cyclic hydrocarbon groups which may optionally have one, two or more singly or doubly unsaturated bonds or one, two or more heteroatoms selected from the group consisting of O, N, P and S. Moreover, the alkyl groups according to the invention may optionally be substituted by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which may have one or more heteroatoms selected from the group consisting of N, O, P and S.

The definition C₁-C₁₂-alkyl comprises the greatest range defined herein for an alkyl group. Specifically, this definition comprises, for example, the meanings methyl, ethyl, n-, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl, n-dodecyl.

In the context of the present invention, alkenyl groups are, unless defined otherwise, straight-chain, branched or cyclic hydrocarbon groups having at least one singly unsaturated bond (double bond) and optionally one, two or more singly or doubly unsaturated bonds or one, two or more heteroatoms selected from the group consisting of O, N, P and S. Moreover, the alkenyl groups according to the invention may optionally be substituted by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which may have one or more heteroatoms selected from the group consisting of N, O, P and S.

The definition C₂-C₁₂-alkenyl comprises the greatest range defined herein for an alkenyl group. Specifically, this definition comprises, for example, the meanings vinyl; allyl (2-propenyl), isopropenyl (1-methylethenyl); but-1-enyl (crotyl), but-2-enyl, but-3-enyl; hex-1-enyl, hex-2-enyl, hex-3-enyl, hex-4-enyl, hex-5-enyl; hept-1-enyl, hept-2-enyl, hept-3-enyl, hept-4-enyl, hept-5-enyl, hept-6-enyl; oct-1-enyl, oct-2-enyl, oct-3-enyl, oct-4-enyl, oct-5-enyl, oct-6-enyl, oct-7-enyl; non-1-enyl, non-2-enyl, non-3-enyl, non-4-enyl, non-5-enyl, non-6-enyl, non-7-enyl, non-8-enyl; dec-1-enyl, dec-2-enyl, dec-3-enyl, dec-4-enyl, dec-5-enyl, dec-6-enyl, dec-7-enyl, dec-8-enyl, dec-9-enyl; undec-1-enyl, undec-2-enyl, undec-3-enyl, undec-4-enyl, undec-5-enyl, undec-6-enyl, undec-7-enyl, undec-8-enyl, undec-9-enyl, undec-10-enyl; dodec-1-enyl, dodec-2-enyl, dodec-3-enyl, dodec-4-enyl, dodec-5-enyl, dodec-6-enyl, dodec-7-enyl, dodec-8-enyl, dodec-9-enyl, dodec-10-enyl, dodec-11-enyl; buta-1,3-dienyl, penta-1,3-dienyl.

In the context of the present invention, alkynyl groups are, unless defined otherwise, straight-chain, branched or cyclic hydrocarbon groups having at least one doubly unsaturated bond (triple bond) and optional one, two or more singly or doubly unsaturated bonds or one, two or more heteroatoms selected from the group consisting of O, N, P and S. Moreover, the alkynyl groups according to the invention may optionally be substituted by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a straight-chain, branched or cyclic C₁₋₁₂-alkyl group which may have one or more heteroatoms selected from the group consisting of N, O, P and S.

The definition C₂-C₁₂-alkynyl comprises the greatest range defined herein for an alkynyl group. Specifically, this definition comprises, for example, the meanings ethynyl (acetylenyl); prop-1-ynyl and prop-2-ynyl.

The definition C₃-C₈-cycloalkyl comprises monocyclic saturated hydrocarbon groups having 3 to 8 carbon ring members, such as cyclopropyl, cyclobutyl, cyclopentyl, cyclohexyl, cycloheptyl and cyclooctyl.

In the context of the present invention, aryl groups are, unless defined otherwise, aromatic hydrocarbon groups which may have one, two or more heteroatoms selected from the group consisting of O, N, P and S and may optionally be substituted by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which may have one or more heteroatoms selected from the group consisting of N, O, P and S.

The definition C₅₋₁₈-aryl comprises the greatest range defined herein for an aryl group having 5 to 18 skeleton atoms, where the carbon atoms may be replaced by heteroatoms. Specifically, this definition comprises, for example, the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In the context of the present invention, arylalkyl groups (aralkyl groups) are, unless defined otherwise, alkyl groups which are substituted by alkyl groups and may have a C₁₋₈-alkylene chain and may be substituted in the aryl skeleton or the alkylene chain by one or more heteroatoms selected from the group consisting of O, N, P and S and optionally by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which may have one or more heteroatoms selected from the group consisting of N, O, P and S.

The definition C₇₋₁₉-aralkyl group comprises the greatest range defined herein for an arylalkyl group having a total of 7 to 19 atoms in the skeleton and the alkylene chain. Specifically, this definition comprises, for example, the meanings benzyl and phenylethyl.

In the context of the present invention, alkylaryl groups (alkaryl groups) are, unless defined otherwise, aryl groups which are substituted by alkyl groups and which may have a C₁₋₈-alkylene chain and may be substituted in the aryl skeleton or the alkylene chain by one or more heteroatoms selected from the group consisting of O, N, P and S and optionally by further groups selected from the group consisting of —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which may have one or more heteroatoms selected from the group consisting of N, O, P and S.

The definition C₇₋₁₉-alkylaryl group comprises the greatest range defined herein for an alkylaryl group having a total of 7 to 19 atoms in the skeleton and the alkylene chain. Specifically, this definition comprises, for example, the meanings tolyl-, 2,3-, 2,4-, 2,5-, 2,6-, 3,4- or 3,5-dimethylphenyl.

The alkyl, alkenyl, alkynyl, aryl, alkaryl and aralkyl groups may additionally have one or more heteroatoms which—unless defined otherwise—are selected from the group consisting of N, O, P and S. Here, the heteroatoms replace the carbon atoms indicated.

The aminothiazoles according to the invention may, if appropriate, be present as mixtures of various possible isomeric forms, in particular stereoisomers such as, for example, E and Z, threo and erythro, and also optical isomers, and, if appropriate, also of tautomers. What is disclosed and claimed is both the E and the Z isomers, and also the threo and erythro, and also the optical isomers, any mixtures of these isomers, and also the possible tautomeric forms.

The aminothiazoles according to the invention are compounds of the formula (I)

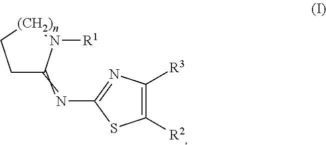

and their salts, N-oxides, metal complexes and stereoisomers.

In formula (I), the groups have the meanings defined below. The given definitions likewise apply to all intermediates:

R¹ is selected from the group consisting of hydrogen; straight-chain or branched C₁₋₁₂-alkyl, C₂₋₁₂-alkenyl, C₂₋₁₂-alkynyl-, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, —COOR′, —COR′, —CONR₂′, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN and —CONR₂′, where R′ is hydrogen or a straight-chain or branched C₁₋₁₂-alkyl, C₁₋₆-haloalkyl or cyclic C₃₋₈-alkyl group which may have 1 to 6 halogen atoms;

R² is selected from the group consisting of hydrogen and straight-chain or branched C₁₋₆-alkyl, C₂₋₁₂-alkenyl, C₂₋₁₂-alkynyl-, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN and —CONR₂′, where R′ has the meanings given above;

R³ is selected from the group consisting of straight-chain or branched C₁₋₆-alkyl, C₂₋₁₂-alkenyl, C₂₋₁₂-alkynyl-, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where in the ring system of all the cyclic groups mentioned above one or more carbon atoms may be replaced by heteroatoms selected from the group consisting of N, O, P and S, and all the groups mentioned above may be substituted by one or more groups selected from the group consisting of —R′, —X, —OR′, —SR′, —NR′₂, —SiR′₃, —COOR′, —CN, C₅₋₁₈-aryl, C₅₋₁₈-aryloxy, C₂₋₁₂-alkenyloxy, C₇₋₁₉-aralkyl and —CONR₂′, where R′ has the meanings given above and the C₅₋₁₈-aryl, C₅₋₁₈-aryloxy, C₂₋₁₂-alkenyloxy and C₇₋₁₉-aralkyl groups may be substituted by one, two or more radicals selected from the group consisting of —R′, —X, —OR′, —SR′, —NR′₂, —SiR′₃, —COOR′, —CN, aryloxy and —CONR₂′, where R′ has the meanings given above;

n is 0, 1 or 2.

In formula (I), the groups have the preferred meanings defined below. The definitions given as being preferred likewise apply to all intermediates:
R¹ is selected from the group consisting of hydrogen, C₁₋₈-alkyl groups, —COOR′, —COR′, —CONR₂′, where R′ has the meanings given above;
R² is selected from the group consisting of hydrogen and straight-chain or branched C₁₋₈-alkyl groups;
R³ is selected from the group consisting of phenyl and phenyl-C₁₋₄-alkyl groups which may be substituted at the phenyl ring by one, two or more halogen atoms, phenyl, phenoxy, C₂₋₆-alkenyloxy, C₁₋₆-alkoxy, phenyl-C₁₋₄-alkyl groups or C₁₋₅-haloalkyl groups or which may be fused with the five-, six- or seven-membered carbocyclic or heterocyclic rings and which may have one or two heteroatoms selected from the group consisting of O and N in the ring, where all the groups mentioned may be substituted by halogen atoms, C₁₋₆-alkyl, C₁₋₆-alkoxy or C₁₋₅-haloalkyl groups;

n is 0 or 1.

In formula (I), the radicals have the particularly preferred meanings defined below. The definitions given as being particularly preferred likewise apply to all intermediates:
R¹ is selected from the group consisting of hydrogen, C₁₋₈-alkyl groups, —COOR′, —COR′, —CONR₂′;
R² is hydrogen;
R³ is selected from
   phenyl, benzyl or phenethyl groups which may be substituted by one, two or more halogen atoms, straight-chain or branched C₁₋₆-alkyl or C₁₋₆-haloalkyl groups or by phenyl or benzyl groups, where the latter phenyl or benzyl groups may be substituted by one, two or more halogen atoms, methyl or methoxy groups;
   phenoxyphenyl or diphenoxyphenyl groups which may be substituted in the phenoxy radical by one or two halogen atoms, straight-chain or branched C₁₋₆-alkyl or C₁₋₆-haloalkyl groups;
   C₂₋₆-alkenyloxyphenyl groups which may be substituted by one or two halogen atoms, straight-chain or branched C₁₋₆-alkyl or C₁₋₆-haloalkyl groups;
   C₁₋₆-alkoxyphenyl groups which may be substituted by one or two halogen atoms, straight-chain or branched C₁₋₆-alkyl or C₁₋₆-haloalkyl groups;
   1,3-benzodioxol-5-yl groups;
   pyridyl groups which may be substituted by one or two halogen atoms, straight-chain or branched C₁₋₆-alkyl or C₁₋₆-haloalkyl groups;
   C₃₋₆-cycloalkyl-C₁₋₄-alkyl groups;

n is 1.

In addition, the present invention also relates to the salts, N-oxides, metal complexes of the aminothiazoles described above and their stereoisomers, in particular to the E and Z isomers with respect to the C═N double bond or mixtures of these.

Depending on the nature of the substituents defined above, the aminothiazoles of the formula (I) have acidic or basic properties and can form salts, if appropriate also inner salts, or adducts with inorganic or organic acids or with bases or with metal ions.

Suitable metal ions are in particular the ions of the elements of the second main group, in particular calcium and magnesium, of the third and fourth main group, in particular aluminum, tin and lead, and also of the first to eighth transition group, in particular chromium, manganese, iron, cobalt, nickel, copper, zinc and others. Particular preference is given to the metal ions of the elements of the fourth period. Here, the metals can be present in the various valencies that they can assume.

If the aminothiazoles of the formula (I) carry hydroxyl, carboxyl or other groups which induce acidic properties, these compounds can be reacted with bases to give salts.

Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and also chlorocholine.

If the aminothiazoles of the formula (I) carry amino, alkylamino or other groups which induce basic properties, these compounds can be reacted with acids to give salts.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄.

Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl groups of 1 to 20 carbon atoms), arylsulphonic acids or -disulphonic acids (aromatic groups, such as phenyl and naphthyl, which carry one or two sulphonic acid groups), alkylphosphonic acids (phosphonic acids having straight-chain or branched alkyl groups of 1 to 20 carbon atoms), arylphosphonic acids or -diphosphonic acids (aromatic radicals, such as phenyl and naphthyl, which carry one or two phosphonic acid groups), where the alkyl or aryl groups may carry further substituents, for example p-toluene sulphonic acid, salicylic acid, p-aminosalicylic acid, 2-phenoxybenzoic acid, 2-acetoxybenzoic acid, saccharin, etc.

The salts obtainable in this manner also have fungicidal properties.

Aminothiazoles of the formula (I) which are particularly preferred in the context of the present invention are selected from the group consisting of: 4-[2-(2,4-dimethylphenoxy)phenyl]-N-[(2E/Z)-piperidin-2-ylidene]-1,3-thiazole-2-amine, 4-(4-chlorophenyl)-N-[(2E/Z)-piperidin-2-ylidene]-1,3-thiazole-2-amine, 4-(4-bromophenyl)-N-[(2E/Z)-piperidin-2-ylidene]-1,3-thiazole-2-amine, 4-(2,4-dichlorophenyl)-N-[(2E/Z)-piperidin-2-ylidene]-1,3-thiazole-2-amine, 4-(4-phenoxyphenyl)-N-[(2E/Z)-piperidin-2-ylidene]-1,3-thiazole-2-amine, 4-[3,5-bis(trifluoromethyl)phenyl]-N-[(2E/Z)-piperidin-2-ylidene]-1,3-thiazole-2-amine, 4-[2-(2,4-difluorophenoxy)phenyl]-N-[(2E/Z)-piperidin-2-ylidene]-1,3-thiazole-2-amine, 4-[2-chloro-3-(trifluoromethyl)phenyl]-N-[(2E/Z)-piperidin-2-yl-idene]-1,3-thiazole-2-amine, 4-[4-(2,4-difluorophenoxy)phenyl]-N-[(2E/Z)-piperidin-2-ylidene]-1,3-thiazole-2-amine, 4-(2-phenoxyphenyl)-N-[(2E/Z)-piperidin-2-ylidene]-1,3-thiazole-2-amine, 4-[2-(2,4-dichlorophenoxy)phenyl]N-[(2E/Z)-piperidin-2-ylidene]-1,3-thiazole-2-amine, 4-[2-(3-tert-butyl-4-chlorophenoxy)phenyl]-N-[(2E/Z)-piperidin-2-ylidene]-1,3-thiazole-2-amine, 4-[2-(3-tert-butylphenoxy)phenyl]-N-[(2E/Z)-piperidin-2-ylidene]-1,3-thiazole-2-amine, 4-{2-[4-chloro-3-(trifluoromethyl)phenoxy]phenyl}-N-[(2E/Z)-piperidin-2-ylidene]-1,3-thiazole-2-amine, N-[(2E/Z)-piperidin-2-ylidene]-4-[2-(trifluoromethoxy)phenyl]-1,3-thiazole-2-amine, 1-[(2E/Z)-2-({4-[2-(2,4-difluorophenoxy)phenyl]-1,3-thiazol-2-yl}imino)piperidin-1-yl]ethanone, 1-[(2E/Z)-2-{[4-(2-phenoxyphenyl)-1,3-thiazol-2-yl]imino}piperidin-1-yl]ethanone, 4-(3,4-difluorophenyl)-N-[(2E/Z)-piperidin-2-ylidene]-1,3-thiazole-2-amine, 4-(2,5-difluorophenyl)-N-[(2E/Z)-piperidin-2-ylidene]-1,3-thiazole-2-amine, 4-(2-chlorophenyl)-N-[(2E/Z)-piperidin-2-ylidene]-1,3-thiazole-2-amine, N-[(2E/Z)-piperidin-2-ylidene]-4-[2-(trifluoromethyl)phenyl]-1,3-thiazole-2-amine, N-[(2E/Z)-piperidin-2-ylidene]-4-[2-(prop-2-en-1-yloxy)phenyl]-1,3-thiazole-2-amine, 4-(2-ethoxyphenyl)-N-[(2E/Z)-piperidin-2-ylidene]-1,3-thiazole-2-amine, 4-(2,3-dichlorophenyl)-N-[(2E/Z)-piperidin-2-ylidene]-1,3-thiazole-2-amine, 4-(2,3-difluorophenyl)-N-[(2E/Z)-piperidin-2-ylidene]-1,3-thiazole-2-amine, 4-[2-fluoro-3-(trifluoromethyl)phenyl]-N-[(2E/Z)-piperidin-2-ylidene]-1,3-thiazole-2-amine, 4-(3,4-dimethoxyphenyl)-N-[(2E/Z)-piperidin-2-ylidene]-1,3-thiazole-2-amine, 4-(1,3-benzodioxol-5-yl)-N-[(2E/Z)-piperidin-2-ylidene]-1,3-thiazole-2-amine, 4-(3,5-difluorophenyl)-N-[(2E/Z)-piperidin-2-ylidene]-1,3-thiazole-2-amine, 4-(3-chloro-4-fluorophenyl)-N-[(2E/Z)-piperidin-2-ylidene]-1,3-thiazole-2-amine, 4-(4-chlorophenyl)-5-methyl-N-[(2E/Z)-piperidin-2-ylidene]-1,3-thiazole-2-amine, 4-(4-tert-butylphenyl)-N-[(2E/Z)-piperidin-2-ylidene]-1,3-thiazole-2-amine, 1-[(2E/Z)-2-{[4-(2-ethoxyphenyl)-1,3-thiazol-2-yl]imino}piperidin-1-yl]ethanone, 1-[(2E/Z)-2-{[4-(4-chlorophenyl)-1,3-thiazol-2-yl]imino}piperidin-1-yl]ethanone, 1-[(2E/Z)-2-{[4-(2,4-dichlorophenyl)-1,3-thiazol-2-yl]imino}piperidin-1-yl]ethanone, 1-[(2E/Z)-2-{[4-(2,3-dichlorophenyl)-1,3-thiazol-2-yl]imino}piperidin-1-yl]ethanone, 1-[(2E/Z)-2-{[4-(2,3-difluorophenyl)-1,3-thiazol-2-yl]imino}piperidin-1-yl]ethanone, 1-[(2E/Z)-2-({4-[2-fluoro-3-(trifluoromethyl)phenyl]-1,3-thiazol-2-yl}imino)piperidin-1-yl]ethanone, 1-[(2E/Z)-2-{[4-(1,3-benzodioxol-5-yl)-1,3-thiazol-2-yl]imino}piperidin-1-yl]ethanone, 1-[(2E/Z)-2-{[4-(3,5-difluoro-phenyl)-1,3-thiazol-2-yl]imino}piperidin-1-yl]ethanone, 1-[(2E/Z)-2-{[4-(3-chloro-4-fluorophenyl)-1,3-thiazol-2-yl]imino}piperidin-1-yl]ethanone, 1-[(2E/Z)-2-{[4-(4-chlorophenyl)-5-methyl-1,3-thiazol-2-yl]imino}piperidin-1-yl]ethanone, 1-[(2E/Z)-2-{[4-(2,3-difluorophenyl)-5-methyl-1,3-thiazol-2-yl]imino}piperidin-1-yl]ethanone, 4-(2,3-difluorophenyl)-5-methyl-N-[(2E/Z)-piperidin-2-ylidene]-1,3-thiazole-2-amine, N-[(2E/Z)-piperidin-2-ylidene]-4-[3-(trifluoromethyl)phenyl]-1,3-thiazole-2-amine, 4-(3-bromophenyl)-N-[(2E/Z)-piperidin-2-ylidene]-1,3-thiazole-2-amine, 4-[3-(2,2-dimethylpropyl)phenyl]-N-[(2E/Z)-piperidin-2-ylidene]-1,3-thiazole-2-amine, 4-(2-bromophenyl)-N-[(2E/Z)-piperidin-2-ylidene]-1,3-thiazole-2-amine, 4-[4-(difluoromethoxy)phenyl]-N-[(2E/Z)-piperidin-2-ylidene]-1,3-thiazole-2-amine, N-[(2E/Z)-piperidin-2-ylidene]-4-[4-(trifluoromethoxy)phenyl]-1,3-thiazole-2-amine, 4-(2,6-dichlorophenyl)-N-[(2E/Z)-piperidin-2-ylidene]-1,3-thiazole-2-amine, 4-(3,4-dichlorophenyl)-N-[(2E/Z)-piperidin-2-ylidene]-1,3-thiazole-2-amine, 4-(2-chloropyridin-3-yl)-N-[(2E/Z)-piperidin-2-ylidene]-1,3-thiazole-2-amine, 4-(4-fluorophenyl)-N-[(2E/Z)-piperidin-2-ylidene]-1,3-thiazole-2-amine, 4-(3,5-dichlorophenyl)-N-[(2E/Z)-piperidin-2-ylidene]-1,3-thiazole-2-amine, 4-(4-methoxyphenyl)-N-[(2E/Z)-piperidin-2-ylidene]-1,3-thiazole-2-amine, 4-(3-methoxyphenyl)-N-[(2E/Z)-piperidin-2-ylidene]-1,3-thiazole-2-amine, 4-(2,6-dimethylpyridin-3-yl)-N-[(2E/Z)-piperidin-2-ylidene]-1,3-thiazole-2-amine, 4-(3-chlorophenyl)-N-[(2E/Z)-piperidin-2-ylidene]-1,3-thiazole-2-amine, 4-(3-methylphenyl)-N-[(2E/Z)-piperidin-2-ylidene]-1,3-thiazole-2-amine, N-[(2E/Z)-piperidin-2-ylidene]-4-(pyridin-2-yl)-1,3-thiazole-2-amine, 4-(6-methylpyridin-2-yl)-N-[(2E/Z)-piperidin-2-ylidene]-1,3-thiazole-2-amine, 4-(6-chloropyridin-3-yl)-N-[(2E/Z)-piperidin-2-ylidene]-1,3-thiazole-2-amine, 4-(biphenyl-2-yl)-N-[(2E/Z)-piperidin-2-ylidene]-1,3-thiazole-2-amine, 4-(3′-methylbiphenyl-2-yl)-N-[(2E/Z)-piperidin-2-ylidene]-1,3-thiazole-2-amine, 4-(3′,4′-dichloro-biphenyl-2-yl)-N-[(2E/Z)-piperidin-2-ylidene]-1,3-thiazole-2-amine, 4-(4′-methoxybiphenyl-2-yl)-N-[(2E/Z)-piperidin-2-ylidene]-1,3-thiazole-2-amine, 4-(4′-methoxybiphenyl-3-yl)-N-[(2E/Z)-piperidin-2-ylidene]-1,3-thiazole-2-amine, 4-(2-chlorobenzyl)-N-[(2E/Z)-piperidin-2-ylidene]-1,3-thiazole-2-amine, 4-(3-chlorobenzyl)-N-[(2E/Z)-piperidin-2-ylidene]-1,3-thiazole-2-amine, 4-(3′-methylbiphenyl-3-yl)-N-[(2E/Z)-piperidin-2-ylidene]-1,3-thiazole-2-amine, 4-(cyclopropylmethyl)-N-[(2E/Z)-piperidin-2-ylidene]-1,3-thiazole-2-amine, 4-(1-phenylethyl)-N-[(2E/Z)-piperidin-2-ylidene]-1,3-thiazole-2-amine, 4-[2-(4-chlorophenoxy)phenyl]-N-[(2E/Z)-piperidin-2-ylidene]-1,3-thiazole-2-amine, 4-[2-(4-fluorophenoxy)phenyl]-N-[(2E/Z)-piperidin-2-ylidene]-1,3-thiazole-2-amine, 4-[2-(2,6-difluorophenoxy)phenyl]-N-[(2E/Z)-piperidin-2-ylidene]-1,3-thiazole-2-amine, N-[(2E/Z)-piperidin-2-ylidene]-4-[2-(2,3,4-trifluorophenoxy)phenyl]-1,3-thiazole-2-amine, 4-[2-(3,4-difluorophenoxy)phenyl]-N-[(2E/Z)-piperidin-2-ylidene]-1,3-thiazole-2-amine, 4-[2-(4-methylphenoxy)phenyl]-N-[(2E/Z)-piperidin-2-ylidene]-1,3-thiazole-2-amine, 4-[2-(2,5-difluorophenoxy)phenyl]-N-[(2E/Z)-piperidin-2-ylidene]-1,3-thiazole-2-amine, 4-(2-benzylphenyl)-N-[(2E/Z)-piperidin-2-ylidene]-1,3-thiazole-2-amine, N-[(2E/Z)-piperidin-2-ylidene]-4-[2-(2,4,6-trifluorophenoxy)phenyl]-1,3-thiazole-2-amine, 4-[2-(3-fluorophenoxy)phenyl]-N-[(2E/Z)-piperidin-2-ylidene]-1,3-thiazole-2-amine, 4-[2-(4-bromophenoxy)phenyl]-N-[(2E/Z)-piperidin-2-ylidene]-1,3-thiazole-2-amine, 4-[2-(3-chlorophenoxy)phenyl]-N-[(2E/Z)-piperidin-2-ylidene]-1,3-thiazole-2-amine, 4-[2-(2-bromophenoxy)phenyl]-N-[(2E/Z)-piperidin-2-ylidene]-1,3-thiazole-2-amine, 4-[2-(3-bromophenoxy)phenyl]-N-[(2E/Z)-piperidin-2-ylidene]-1,3-thiazole-2-amine, 4-[2-(2,4-difluorobenzyl)phenyl]-N-[(2E/Z)-piperidin-2-ylidene]-1,3-thiazole-2-amine, 4-[2-(3,5-dichlorobenzyl)phenyl]-N-[(2E/Z)-piperidin-2-ylidene]-1,3-thiazole-2-amine, 4-[2-(4-fluorobenzyl)phenyl]-N-[(2E/Z)-piperidin-2-ylidene]-1,3-thiazole-2-amine, 4-[2-(2,6-dichlorobenzyl)phenyl]-N-[(2E/Z)-piperidin-2-ylidene]-1,3-thiazole-2-amine, 4-[2-(3,4-dichloro-benzyl)phenyl]-N-[(2E/Z)-piperidin-2-ylidene]-1,3-thiazole-2-amine, 4-[2-(4-chlorobenzyl)-phenyl]-N-[(2E/Z)-piperidin-2-ylidene]-1,3-thiazole-2-amine, 4-(4-benzylphenyl)-N-[(2E/Z)-piperidin-2-ylidene]-1,3-thiazole-2-amine, 4-[2-(2,4-dimethylphenoxy)phenyl]-N-[(2E/Z)-pyrrolidin-2-ylidene]-1,3-thiazole-2-amine, 4-(4-chlorophenyl)-N-[(2E/Z)-pyrrolidin-2-ylidene]-1,3-thiazole-2-amine, 4-[4-(2,4-difluorophenoxy)phenyl]-N-[(2E/Z)-pyrrolidin-2-ylidene]-1,3-thiazole-2-amine, 4-(2-phenoxyphenyl)-N-[(2E/Z)-pyrrolidin-2-ylidene]-1,3-thiazole-2-amine, 4-[2-(2,4-dichlorophenoxy)phenyl]-N-[(2E/Z)-pyrrolidin-2-ylidene]-1,3-thiazole-2-amine, 4-[2-(3-tert-butylphenoxy)phenyl]-N-[(2E/Z)-pyrrolidin-2-ylidene]-1,3-thiazole-2-amine, 4-[2-(3-tert-butyl-4-chlorophenoxy)phenyl]-N-[(2E/Z)-pyrrolidin-2-ylidene]-1,3-thiazole-2-amine, 4-[2-(2,4-difluorophenoxy)phenyl]-N-[(2E/Z)-pyrrolidin-2-ylidene]-1,3-thiazole-2-amine, 4-(4-chlorophenyl)-N-[(2E/Z)-piperidin-2-ylidene]-1,3-thiazole-2-amine, 4-(2,4-dichlorophenyl)-N-[(2E/Z)-piperidin-2-ylidene]-1,3-thiazole-2-amine, N-[(2E/Z)-piperidin-2-ylidene]-4-[3-(trifluoromethyl)phenyl]-1,3-thiazole-2-amine, 4-(3-bromophenyl)-N-[(2E/Z)-piperidin-2-ylidene]-1,3-thiazole-2-amine, 4-(2-bromophenyl)-N-[(2E/Z)-piperidin-2-ylidene]-1,3-thiazole-2-amine.

In a preferred embodiment of the present invention, the following compounds are excluded from the formula (I) (or formula (Ia) or formula (Ib)):
compounds of the formula (I) in which n=0, R¹=methyl, R²=hydrogen, R³=phenyl, methyl or 4-bromophenyl (compounds 6a-6c; Liebscher et al, Synthesis, Georg Thieme Verlag, Stuttgart, DE, Vol. 12, 1 Jan. 1989, p. 970),
compound of the formula (I) in which n=0, R¹=hydrogen, R²=hydrogen, R³=4-fluorophenyl (Database Registry, Chemical Abstracts Service, Database accession no. 300851-72-3; XP002563997; Database Chemcats, Database accession no. 2086369943; XP002564129),
compound of the formula (I) in which n=0, R¹=hydrogen, R²=methyl, R³=4-OH-phenyl (Database Registry, Chemical Abstracts Service, Database accession no. 328039-30-1; XP002564118),
compound of the formula (I) in which n=0, R¹=hydrogen, R²=methyl, R³=4-EtO-phenyl (Database Registry, Chemical Abstracts Service, Database accession no. 300568-62-1; XP002564122),
compound of the formula (I) in which n=0, R¹=hydrogen, R²=hydrogen, R³=4-F₂CH—O-phenyl (Database Registry, Chemical Abstracts Service, Database accession no. 380183-46-0; XP002564124),
compound of the formula (I) in which n=0, R¹=hydrogen, R²=hydrogen, R³=4-bromophenyl (Database Registry, Chemical Abstracts Service, Database accession no. 377760-05-9; XP002564125),
compound of the formula (I) in which n=0, R¹=hydrogen, R²=hydrogen, R³=tert-butyl (Database Registry, Chemical Abstracts Service, Database accession no. 753466-17-0; XP002564128),
compound of the formula (I) in which n=0, R¹=hydrogen, R²=hydrogen, R³=methyl (Database Registry, Chemical Abstracts Service, Database accession no. 753465-01-9; XP002565543),
compound of the formula (I) in which n=0, R¹=hydrogen, R²=hydrogen, R³=4-Me-O-phenyl (Database Registry, Chemical Abstracts Service, Database accession no. 887203-42-1 and 887203-39-6; XP002564134; XP002565544),
compound of the formula (I) in which n=0, R¹=hydrogen, R²=hydrogen, R³=4-Me-phenyl (Database Registry, Chemical Abstracts Service, Database accession no. 887203-36-3; XP002565545),
compound of the formula (I) in which n=0, R¹=hydrogen, R²=hydrogen, R³=phenyl (Database Registry, Chemical Abstracts Service, Database accession no. 887203-33-0; XP002565546).

In a further preferred embodiment of the present invention, the following compounds are excluded from the formula (IV):
compound of the formula (IV) in which R²=hydrogen and R³=—CH₂COOH (Aldrich “Catalogue Handbook of Fine Chemicals” 1990, Aldrich, Brussels, p. 87.
compound of the formula (IV) in which R²=—[CH₂]_{(3,4 or 5)}CO₂H and R³=methyl (Swain G; Journal of the Chemical Society, London England, 1 Jan. 1949),
compound of the formula (IV) in which R²=methyl and R³=methyl (Prithwi Nath et al, Journal of the Chemical Society, Calcutta, Ind, Vol. 37, No. 4, 1 Jan. 1960),
compounds of the formula (IV) in which R²=phenyl or hydrogen and R³=styryl (Southwick, Sapper; Journal of Organic Chemistry, Vol. 19, 1954),
compounds of the formula (IV) in which R²=hydrogen and R³=n-butyl (Erlenmeyer H et al., Helvetica Chimica Acta, Verlag Helvetica Chimica Acta, Basle, CH, Vol. 32, No. 1; 1 Feb. 1949).

### Preparation of the Aminothiazoles of the Formula (I) According to the Invention

The aminothiazoles according to the invention can be obtained, for example, by the process according to Scheme 1 below.

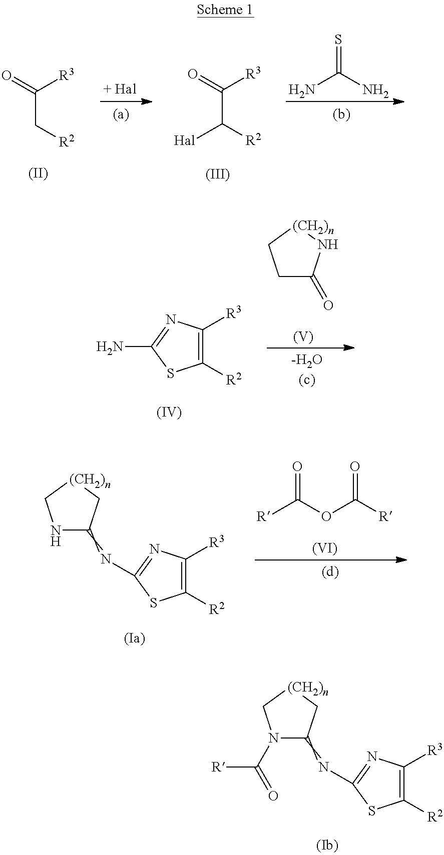

The process steps (a) and (b) shown in Scheme 1 have already been described in the prior art, for example in Molecules, 2003, 8, 793-865 (a) or T. M. Potewar et al in Tetrahedron 64 (2008) 5019-5022 (b). The present invention furthermore provides the conversion of the 2-aminothiazoles of the formula (IV) into the compounds of the formula (VI) in step (c) and their subsequent conversion into the substituted thiazoles of the formula (I) according to step (d).

Accordingly, the process according to the invention comprises at least one of the partial steps (c) and (d); preferably, the process comprises at least the combination of partial steps (c) and (d).

The individual reaction steps are illustrated in more detail below:

### Step (a) Preparation of the α-Haloketones of the Formula (III)

The synthesis of the α-haloketones of the formula (III) can be carried out by halogenation of α-methyleneketones of the formula II; the reaction has already been described in the prior art, for example in Molecules, 2003, 8, 793-865. The halogen atom (Hal) is selected from the group consisting of chlorine and bromine; preferably Hal=bromine.

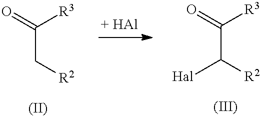

The halogenation can be carried out using the customary halogenating agents; Cl₂ and Br₂, PCl₅, PCl₃, POCl₃, SO₂Cl₂ and SOCl₂ may be mentioned by way of example.

The halogenation of the α-methyleneketones of the formula II can be carried out in the absence of a solvent or in a solvent; preferably, the reaction is carried out in a solvent selected from customary solvents which are inert at the prevailing reaction conditions.

Preference is given to using aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide, alcohols when using SO₂Cl₂, such as, for example, methanol, ethanol or mixtures of these.

The reaction can be carried out under reduced pressure, at atmospheric pressure or under superatmospheric pressure and at temperatures of from −20 to 200° C.; preferably, the reaction is carried out at atmospheric pressure and temperatures of from 50 to 150° C.

### Step (b) Synthesis of the 2-Aminothiazoles of the Formula (IV)

The α-haloketones of the formula III are reacted with thiourea to give the 2-aminothiazoles of the formula (IV).

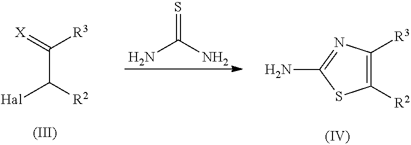

The reaction of the α-haloketones (III) with thiourea can be carried out in the absence of a solvent or in a solvent; preferably, the reaction is carried out in a solvent selected from customary solvents which are inert at the prevailing reaction conditions.

Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide; or mixtures of these with water, and also pure water.

The reaction can be carried out under reduced pressure, at atmospheric pressure or under superatmospheric pressure and at temperatures of from −20 to 200° C.; preferably, the reaction is carried out at atmospheric pressure and temperatures of from 50 to 150° C.

Suitable processes for preparing 2-aminothiazoles have already been described in the prior art, for example by T. M. Potewar et al in Tetrahedron 64 (2008) 5019-5022.

Step (c) Reaction of the 2-Aminothiazoles of the Formula (IV) with Lactams of the Formula (V)

The reaction of the 2-aminothiazoles of the formula (IV) with lactams of the formula (V), where n has the meanings described above, in the presence of suitable condensing agents leads to compounds of the formula (Ia).

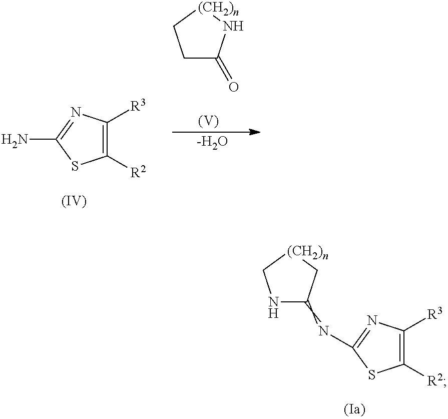

Suitable condensing agents are all reagents which remove water from the reaction mixture and thus shift the equilibrium of the reaction to the side of the product (Ia). Acid halide formers, such as, for example, phosgene, phosphorus tribromide, phosphorus trichloride, phosphorus pentachloride, phosphorus oxychloride or thionyl chloride; anhydride formers, such as, for example, methyl chloroformate, ethyl chloroformate, isopropyl chloroformate, isobutyl chloroformate or methanesulfonyl chloride; carbodiimines, such as, for example, N,N′-dicyclohexylcarbodiimine (DCC) or other customary condensing agents, such as, for example, phosphorus pentoxide, polyphosphoric acid, N,N′-carbodiimidazole, 2-ethoxy-N-ethoxycarbonyl-1,2-dihydroquinoline (EEDQ), triphenylphosphine/carbon tetrachloride or bromotripyrrolidinophosphonium hexafluoro-phosphate may be mentioned by way of example. Particular preference is given to using POCl₃.

The reaction according to step (c) is preferably carried out in a solvent selected from customary solvents which are inert at the prevailing reaction conditions. Preference is given to aliphatic, alicyclic or aromatic hydrocarbons, such as, for example, petroleum ether, hexane, heptane, cyclohexane, methylcyclohexane, benzene, toluene, xylene or decaline; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole.

The reaction can be carried out under reduced pressure, at atmospheric pressure or under superatmospheric pressure and at temperatures of from −20 to 200° C.; preferably, the reaction is carried out at atmospheric pressure and temperatures of from 50 to 150° C.

Particular preference is given to using lactams of the formula (V) selected from the group consisting of pyrrolidone and piperidone.

Step (d) Reaction of Compounds of the Formula (Ia) with Carboxylic Anhydrides of the Formula (VI)

The conversion of the N—H-lactams of the formula (Ia) into N-acetyl-lactams of the formula (Ib) can by carried out by reaction with a carboxylic anhydride of the formula (VI).

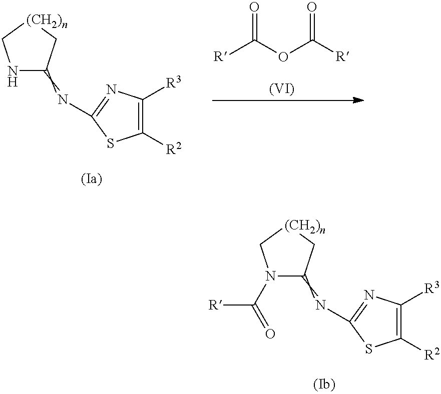

The reaction of the compound (Ia) with a carboxylic anhydride of the formula (VI) can be carried out in the absence of a solvent or in a solvent; preferably, the reaction is carried out in a solvent selected from customary solvents which are inert at the prevailing reaction conditions.

Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylenephosphoric triamide.

The reaction can be carried out under reduced pressure, at atmospheric pressure or under superatmospheric pressure and at temperatures of from −20 to 200° C.; preferably, the reaction is carried out at atmospheric pressure and temperatures of from 50 to 150° C.

Particular preference is given to using carboxylic anhydrides selected from the group consisting of acetic anhydride and propionic anhydride.

### Control of Unwanted Microorganisms

The amidines according to the invention have a potent microbicidal activity and can be employed for controlling undesirable microorganisms, such as fungi and bacteria, in crop protection and in the protection of materials.

### Crop Protection

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

diseases caused by powdery mildew pathogens, such as, for example,

Blumeria species, such as, for example, Blumeria graminis;
Podosphaera species, such as, for example, Podosphaera leucotricha;
Sphaerotheca species, such as, for example, Sphaerotheca fuliginea;
Uncinula species, such as, for example, Uncinula necator;
diseases caused by rust disease pathogens, such as, for example,
Gymnosporangium species, such as, for example, Gymnosporangium sabinae
Hemileia species, such as, for example, Hemileia vastatrix;
Phakopsora species, such as, for example, Phakopsora pachyrhizi and Phakopsora meibomiae;
Puccinia species, such as, for example, Puccinia recondita;
Uromyces species, such as, for example, Uromyces appendiculatus;
diseases caused by pathogens from the group of the Oomycetes, such as, for example,
Bremia species, such as, for example, Bremia lactucae;
Peronospora species, such as, for example, Peronospora pisi or P. brassicae;
Phytophthora species, such as, for example, Phytophthora infestans;
Plasmopara species, such as, for example, Plasmopara viticola;
Pseudoperonospora species, such as, for example, Pseudoperonospora humuli or Pseudoperonospora cubensis;
Pythium species, such as, for example, Pythium ultimum;
leaf blotch diseases and leaf wilt diseases caused, for example, by
Alternaria species, such as, for example, Alternaria solani;
Cercospora species, such as, for example, Cercospora beticola;
Cladosporium species, such as, for example, Cladosporium cucumerinum;
Cochliobolus species, such as, for example, Cochliobolus sativus (conidia form: Drechslera, syn: Helminthosporium);
Colletotrichum species, such as, for example, Colletotrichum lindemuthanium;
Cycloconium species, such as, for example, Cycloconium oleaginum;
Diaporthe species, such as, for example, Diaporthe citri;
Elsinoe species, such as, for example, Elsinoe fawcettii;
Gloeosporium species, such as, for example, Gloeosporium laeticolor;
Glomerella species, such as, for example, Glomerella cingulata;
Guignardia species, such as, for example, Guignardia bidwelli;
Leptosphaeria species, such as, for example, Leptosphaeria maculans;
Magnaporthe species, such as, for example, Magnaporthe grisea;
Mycosphaerella species, such as, for example, Mycosphaerella graminicola and Mycosphaerella fijiensis;
Phaeosphaeria species, such as, for example, Phaeosphaeria nodorum;
Pyrenophora species, such as, for example, Pyrenophora teres;
Ramularia species, such as, for example, Ramularia collo-cygni;
Rhynchosporium species, such as, for example, Rhynchosporium secalis;
Septoria species, such as, for example, Septoria apii;
Typhula species, such as, for example, Typhula incarnata;
Venturia species, such as, for example, Venturia inaequalis;
root and stem diseases caused, for example, by
Corticium species, such as, for example, Corticium graminearum;
Fusarium species, such as, for example, Fusarium oxysporum;
Gaeumannomyces species, such as, for example, Gaeumannomyces graminis;
Rhizoctonia species, such as, for example, Rhizoctonia solani;
Tapesia species, such as, for example, Tapesia acuformis;
Thielaviopsis species, such as, for example, Thielaviopsis basicola;
ear and panicle diseases (including corn cobs) caused, for example, by
Alternaria species, such as, for example, Alternaria spp.;
Aspergillus species, such as, for example, Aspergillus flavus;
Cladosporium species, such as, for example, Cladosporium cladosporioides;
Claviceps species, such as, for example, Claviceps purpurea;
Fusarium species, such as, for example, Fusarium culmorum;
Gibberella species, such as, for example, Gibberella zeae;
Monographella species, such as, for example, Monographella nivalis;
diseases caused by smut fungi, such as, for example,
Sphacelotheca species, such as, for example, Sphacelotheca reiliana;
Tilletia species, such as, for example, Tilletia caries;
Urocystis species, such as, for example, Urocystis occulta;
Ustilago species, such as, for example, Ustilago nuda;
fruit rot caused, for example, by
Aspergillus species, such as, for example, Aspergillus flavus;
Botrytis species, such as, for example, Botrytis cinerea;
Penicillium species, such as, for example, Penicillium expansum and Penicillium purpurogenum;
Sclerotinia species, such as, for example, Sclerotinia sclerotiorum;
Verticilium species, such as, for example, Verticilium alboatrum;
seed- and soil-borne rot and wilt diseases, and also diseases of seedlings, caused, for example, by
Alternaria species, such as, for example, Alternaria brassicicola;
Aphanomyces species, such as, for example, Aphanomyces euteiches;
Ascochyta species, such as, for example, Ascochyta lentis;
Aspergillus species, such as, for example, Aspergillus flavus;
Cladosporium species, such as, for example, Cladosporium herbarum;
Cochliobolus species, such as, for example, Cochliobolus sativus; (conidia form: Drechslera, Bipolaris Syn: Helminthosporium);
Colletotrichum species, such as, for example, Colletotrichum coccodes;
Fusarium species, such as, for example, Fusarium culmorum;
Gibberella species, such as, for example, Gibberella zeae;
Macrophomina species, such as, for example, Macrophomina phaseolina;
Monographella species, such as, for example, Monographella nivalis;
Penicillium species, such as, for example, Penicillium expansum;
Phoma species, such as, for example, Phoma lingam;
Phomopsis species, such as, for example, Phomopsis sojae;
Phytophthora species, such as, for example, Phytophthora cactorum;
Pyrenophora species, such as, for example, Pyrenophora graminea;
Pyricularia species, such as, for example, Pyricularia oryzae;
Pythium species, such as, for example, Pythium ultimum;
Rhizoctonia species, such as, for example, Rhizoctonia solani;
Rhizopus species, such as, for example, Rhizopus oryzae
Sclerotium species, such as, for example, Sclerotium rolfsii;
Septoria species, such as, for example, Septoria nodorum;
Typhula species, such as, for example, Typhula incarnata;
Verticillium species, such as, for example, Verticillium dahliae
cancerous diseases, galls and witches' broom caused, for example, by
Nectria species, such as, for example, Nectria galligena;
wilt diseases caused, for example, by
Monilinia species, such as, for example, Monilinia laxa;
deformations of leaves, flowers and fruits caused, for example, by
Taphrina species, such as, for example, Taphrina deformans;
degenerative diseases of woody plants caused, for example, by
Esca species, such as, for example, Phaeomoniella chlamydospora and Phaeoacremonium aleophilum and Fomitiporia mediterranea;
diseases of flowers and seeds caused, for example, by
Botrytis species, such as, for example, Botrytis cinerea;
diseases of plant tubers caused, for example, by
Rhizoctonia species, such as, for example, Rhizoctonia solani;
Helminthosporium species, such as, for example, Helminthosporium solani;
diseases caused by bacterial pathogens, such as, for example,
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
preference is given to controlling the following diseases of soybeans:
fungal diseases on leaves, stems, pods and seeds caused, for example, by

alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola).

Fungal diseases on roots and the stem base caused, for example, by

black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also exhibit a potent strengthening effect in plants. Accordingly, they can be used for mobilizing the defences of the plant against attack by undesirable microorganisms.

Plant-strengthening (resistance-inducing) substances are to be understood as meaning, in the present context, those substances which are capable of stimulating the defence system of plants in such a way that the treated plants, when subsequently inoculated with undesired microorganisms, develop a high degree of resistance to these microorganisms.

In the present case, undesirable microorganisms are to be understood as meaning phytopathogenic fungi, bacteria and viruses. Accordingly, the substances according to the invention can be used to protect plants for a certain period after the treatment against attack by the pathogens mentioned. The period within which protection is brought about generally extends from 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed particularly successfully for controlling cereal diseases such as, for example, against Puccinia species and diseases in viticulture and fruit and vegetable growing such as, for example, against Botrytis, Venturia or Alternaria species.

The active compounds according to the invention are also suitable for increasing the yield of crops. In addition, they show reduced toxicity and are well tolerated by plants.

If appropriate, the active compounds according to the invention can also be employed in specific concentrations and application rates as herbicides, for influencing plant growth, and for controlling animal pests. If appropriate, they can also be used as intermediates and precursors for the synthesis of further active compounds.

All plants and plant parts can be treated in accordance with the invention. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. Parts of plants also include harvested material and vegetative and generative propagation material, for example seedlings, tubers, rhizomes, cuttings and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

### Mycotoxins

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins which may be produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

### Protection of Materials

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae. Microorganisms of the following genera may be mentioned as examples:

Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

### Formulations

The present invention relates to a composition for controlling unwanted microorganisms which comprises at least one of the isothiazolyloxyphenylamidines according to the invention.

To this end, depending on their particular physical and/or chemical properties, the isothiazolyloxyphenylamidines according to the invention can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. As solid carriers these are suitable: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite, dolomite, and synthetic granules of inorganic and organic meals, and also granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. As dispersants there are suitable: for example lignosulphite waste liquors and methylcellulose.

Tackifiers such as carboxymethylcellulose and natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The formulations described above can be used in a method according to the invention for controlling unwanted microorganisms, where the isothiazolyloxyphenylamidines according to the invention are applied to the microorganisms and/or to their habitat.

### Treatment of Seed

The control of phytopathogenic fungi by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with, or at least reduce considerably, the additional application of crop protection agents after planting or after emergence of the plants. It is furthermore desirable to optimize the amount of active compound employed in such a way as to provide optimum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular relates to a method for the protection of seed and germinating plants from attack by phytopathogenic fungi, by treating the seed with a composition according to the invention.

The invention also relates to the use of the compositions according to the invention for treating seed for protecting the seed and the germinating plant against phytopathogenic fungi.

Furthermore, the invention relates to seed treated with a composition according to the invention for protection against phytopathogenic fungi.

One of the advantages of the present invention is that the particular systemic properties of the compositions according to the invention mean that treatment of the seed with these compositions not only protects the seed itself, but also the resulting plants after emergence, from phytopathogenic fungi. In this manner, the immediate treatment of the crop at the time of sowing or shortly thereafter can be dispensed with.

It is likewise to be considered advantageous that the mixtures according to the invention can be used in particular also for transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soybeans, rice, potatoes, sunflowers, beans, coffee, beets (for example sugarbeets and fodder beets), peanuts, vegetables (such as tomatoes, cucumbers, onions and lettuce), lawns and ornamental plants. The treatment of the seed of cereals (such as wheat, barley, rye and oats), maize and rice is of particular importance.

Within the context of the present invention, the composition according to the invention is applied to the seed either alone or in a suitable formulation. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, it is possible to use, for example, seed which has been harvested, cleaned and dried to a moisture content of less than 15% by weight. Alternatively, it is also possible to use seed which, after drying, has been treated, for example, with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which can have phytotoxic effects at certain application rates.

The compositions according to the invention can be applied directly, i.e. without containing any other components and undiluted. In general, it is preferred to apply the compositions to the seed in the form of a suitable formulation. Suitable formulations and methods for treating seed are known to the person skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations which can be used in accordance with the invention can be converted into the customary seed-dressing formulations, such as solutions, emulsions, suspensions, powders, foams, slurries or other coating compositions for seed, and also ULV formulations.

These formulations are prepared in a known manner, by mixing the active compounds or active compound combinations with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. In this context, not only pigments, which are sparingly soluble in water, but also dyes, which are soluble in water, may be used. Examples which may be mentioned are the colorants known by the names Rhodamin B, C.I. Pigment Red 112 and C.I. Solvent Red 1.

Suitable wetting agents which may be present in the seed-dressing formulations which can be used in accordance with the invention are all substances which promote wetting and which are conventionally used for the formulation of agrochemical active compounds. Preference is given to using alkylnaphthalenesulphonates, such as diisopropyl- or diisobutylnaphthalenesulphonates.

Suitable dispersants and/or emulsifiers which may be present in the seed-dressing formulations which can be used in accordance with the invention are all nonionic, anionic and cationic dispersants conventionally used for the formulation of agrochemical active compounds. Preference is given to using nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristryrylphenol polyglycol ether, and their phosphated or sulphated derivatives. Suitable anionic dispersants are, in particular, lignosulphonates, polyacrylic acid salts and arylsulphonate/formaldehyde condensates.

Antifoams which may be present in the seed-dressing formulations which can be used in accordance with the invention are all foam-inhibiting substances conventionally used for the formulation of agrochemical active compounds. Silicone antifoams and magnesium stearate can preferably be used.

Preservatives which may be present in the seed-dressing formulations which can be used in accordance with the invention are all substances which can be employed for such purposes in agrochemical compositions. Dichlorophene and benzyl alcohol hemiformal may be mentioned by way of example.

Secondary thickeners which may be present in the seed-dressing formulations which can be used in accordance with the invention are all substances which can be employed for such purposes in agrochemical compositions. Cellulose derivatives, acrylic acid derivatives, xanthan, modified clays and finely divided silica are preferred.

Adhesives which may be present in the seed-dressing formulations which can be used in accordance with the invention are all customary binders which can be employed in seed-dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schädlingsbekämpfungsmittel” [Chemistry of plant protection agents and pesticides], vol. 2, Springer Verlag, 1970, p. 401-412).

The seed-dressing formulations which can be used in accordance with the invention can be employed for the treatment of a wide range of seed, either directly or after previously having been diluted with water. Thus, the concentrates or the preparations obtainable therefrom by dilution with water may be used to dress the seed of cereals, such as wheat, barley, rye, oats, and triticale, and also the seed of maize, rice, oilseed rape, peas, beans, cotton, sunflowers, and beets, or else vegetable seed of any of a very wide variety of kinds. The seed-dressing formulations which can be used according to the invention or their dilute preparations may also be used to dress seed of transgenic plants. In this context, additional synergistic effects may also occur in cooperation with the substances formed by expression.

All mixers which can conventionally be employed for the seed-dressing operation are suitable for treating seed with the seed-dressing formulations which can be used in accordance with the invention or with the preparations prepared therefrom by addition of water. Specifically, a procedure is followed during the seed-dressing operation in which the seed is placed into a mixer, the specific desired amount of seed-dressing formulations, either as such or after previously having been diluted with water, is added, and everything is mixed until the formulation is distributed uniformly on the seed. If appropriate, this is followed by a drying process.

The application rate of the seed dressing formulations which can be used according to the invention may be varied within a relatively wide range. It depends on the respective content of the active compounds in the formulations and on the seed. The active compound combination application rates are generally between 0.001 and 50 g per kilogram of seed, preferably between 0.01 and 15 g per kilogram of seed.

Mixtures with Known Fungicides, Bactericides, Acaricides, Nematicides or Insecticides

The amidines according to the invention can be used as such or in their formulations, also in a mixture with known fungicides, bactericides, acaricides, nematicides or insecticides, to broaden, for example, the activity spectrum or to prevent development of resistance.

A mixture with other known active compounds, such as herbicides, or with fertilizers and growth regulators, safeners and/or semiochemicals is also possible.

In addition, the aminothiazoles of the formula (I) according to the invention also have very good antimycotic activity. They have a very broad antimycotic activity spectrum in particular against dermatophytes and yeasts, molds and diphasic fungi, (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

Accordingly, the isothiazolyloxyphenylamidines according to the invention can be used both in medical and in non-medical applications.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil.

It is also possible to treat the seed of the plants.

When using the isothiazolyloxyphenylamidines according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. In the treatment of the soil, the application rates of active compound are generally between 0.1 and 10 000 g/ha, preferably between 1 and 5000 g/ha.

### GMOs

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNAi technology [RNA interference]). A heterologous gene that is located in the genome is also called a transgene. A transgene that is defined by its particular location in the plant genome is called a transformation or transgenic event.

Depending on the plant species or plant varieties, their location and growth conditions (soils, climate, vegetation period, diet), the treatment according to the invention may also result in superadditive (“synergistic”) effects. Possible are thus, for example, the following effects which exceed the effects which are to be expected: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

At certain application rates, the active compound combinations according to the invention may also have a strengthening effect in plants. Accordingly, they are suitable for mobilizing the defence system of the plant against attack by unwanted phytopathogenic fungi and/or microorganisms and/or viruses. This may, if appropriate, be one of the reasons for the enhanced activity of the combinations according to the invention, for example against fungi. Plant-strengthening (resistance-inducing) substances are to be understood as meaning, in the present context, also those substances or combinations of substances which are capable of stimulating the defence system of plants in such a way that, when subsequently inoculated with unwanted phytopathogenic fungi and/or microorganisms and/or viruses, the treated plants display a substantial degree of resistance to these unwanted phytopathogenic fungi and/or microorganisms and/or viruses. In the present case, unwanted phytopathogenic fungi and/or microorganisms and/or viruses are understood as meaning phytopathogenic fungi, bacteria and viruses. Thus, the substances according to the invention can be employed for protecting plants against attack by the abovementioned pathogens within a certain period of time after the treatment. The period within which protection is brought about generally extends from 1 to 10 days, preferably 1 to 7 days, after the treatment of the plants with the active compounds.

Plants and plant varieties which are preferably treated according to the invention include all plants which have genetic material which imparts particularly advantageous, useful traits to these plants (whether obtained by breeding and/or biotechnological means).

Plants and plant varieties which are also preferably treated according to the invention are resistant against one or more biotic stresses, i.e. said plants have a better defence against animal and microbial pests, such as against nematodes, insects, mites, phytopathogenic fungi, bacteria, viruses and/or viroids.

Plants and plant varieties which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or hybrid vigour, which results in generally higher yield, increased vigour, better health and better resistance towards biotic and abiotic stress factors. Such plants are typically made by crossing an inbred male-sterile parent line (the female parent) with another inbred male-fertile parent line (the male parent). Hybrid seed is typically harvested from the male-sterile plants and sold to growers. Male-sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar.

Plants or plant varieties (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants naturally-occurring mutations of the above-mentioned genes.

Other herbicide-resistant plants are for example plants which have been made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Further herbicide-resistant plants are plants that have been made tolerant to acetolactate synthase (ALS) inhibitors. Known ALS inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry 1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR 604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:
a. plants which contain a transgene capable of reducing the expression and/or the activity of the poly(ADP-ribose)polymerase (PARP) gene in the plant cells or plants;
b. plants which contain a stress tolerance-enhancing transgene capable of reducing the expression and/or the activity of the PARG encoding genes of the plants or plants cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant varieties (obtained by plant biotechnology methods such as genetic engineering) which may also be treated according to the invention show altered quantity, quality and/or storage-stability of the harvested product and/or altered properties of specific ingredients of the harvested product such as, for example:
1) Transgenic plants which synthesize a modified starch which is altered with respect to its chemophysical traits, in particular the amylose content or the amylose/amylopectin ratio, the degree of branching, the average chain length, the distribution of the side chains, the viscosity behaviour, the gel resistance, the grain size and/or grain morphology of the starch in comparison to the synthesized starch in wild-type plant cells or plants, such that this modified starch is better suited for certain applications.
2) Transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) Transgenic plants which produce hyaluronan.

Plants or plant varieties (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fiber-selective β-1,3-glucanase;
f) plants, such as cotton plants, which have fibers with altered reactivity, for example through the expression of the N-acetylglucosaminetransferase gene including nodC and chitin synthase genes.

Plants or plant cultivars (that have been obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins are the transgenic plants available under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are available under the following trade names: Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, and that are listed for example in the databases for various national or regional regulatory agencies (see for example http://gmoinfo.jrc.it/gmp_browse.aspx and http://www.agbios.com/dbase.php).

### Preparation Examples

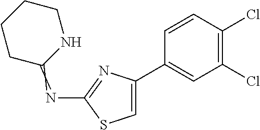

### Synthesis Example 52

0.34 g (3.26 mmol) of valerolactam is initially charged in 15 ml of absolute 1,2-dichloroethane, and 0.30 g (1.96 mmol) of phosphorus oxychloride is added dropwise at room temperature. Over a period of 2 hours, 0.40 g (1.63 mmol) of 4-(3,4-dichlorophenyl)-1,3-thiazole-2-amine is then added at room temperature, and the mixture is heated at the boil under reflux for 16 hours. After cooling, 1N sodium hydroxide solution is added at room temperature until a pH of 14 is reached. Ethyl acetate is added to the syrup-like mixture, which is then stirred until it is once more highly liquid. Extraction with ethyl acetate, drying, concentration and purification on silica gel (cyclohexane/ethyl acetate) gives 0.21 g (0.64 mmol, 39% of theory) of 4-(3,4-dichlorophenyl)-N-[2-piperidin-2-ylidene]-1,3-thiazole-2-amine as a light-yellow solid.

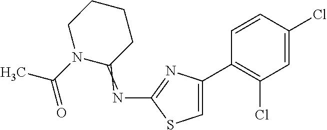

### Synthesis Example 35

0.163 g (0.50 mmol) of 4-(2,4-dichlorophenyl)-N-[2-piperidin-2-ylidene]-1,3-thiazole-2-amine is initially charged in 10 ml of dichloromethane, and 0.152 g (1.50 mmol) of triethylamine is added. At 0° C., 0.133 g (1.30 mmol) acetic anhydride is then added dropwise, and the mixture is stirred at room temperature for 18 hours. For work-up, the mixture is diluted with dichloromethane, quenched with 10% strength citric acid solution, washed with 1N aqueous sodium hydroxide solution, dried and concentrated. This gives 0.230 g (85% of theory) of 1-[2-{[4-(2,4-dichlorophenyl)-1,3-thiazol-2-yl]imino}piperidin-1-yl]ethanone as an oil.

TABLE I

| Ex. | n | R¹ | R² | R³ | log P |
|---|---|---|---|---|---|
| 1 | 2 | H | H | 2-(2,4-dimethylphenoxy)phenyl | 2.66**; 4.9*** |
| 2 | 2 | H | H | 4-chlorophenyl | 1.7** |
| 3 | 2 | H | H | 4-bromophenyl | 1.67** |
| 4 | 2 | H | H | 2,4-dichlorophenyl | 1.8** |
| 5 | 2 | H | H | 4-phenoxyphenyl | 2.17**; 3.97*** |
| 6 | 2 | H | H | 3,5-bis(trifluoromethyl)phenyl | 2.23**; 4.36*** |
| 7 | 2 | H | H | 2-(2,4-difluorophenoxy)phenyl | 2.11**; 3.9***; 2.11* |
| 8 | 2 | H | H | 2-chloro-3-(trifluoromethyl)phenyl | 1.94**; 3.72*** |
| 9 | 2 | H | H | 4-(2,4-difluorophenoxy)phenyl | 3.95***; 2.12* |
| 10 | 2 | H | H | 2-phenoxyphenyl | 2.09**; 3.91***; 2.07* |
| 11 | 2 | H | H | 2-(2,4-dichlorophenoxy)phenyl | 2.42**; 4.84***; 2.42* |
| 12 | 2 | H | H | 2-(3-tert-butyl-4-chlorophenoxy)phenyl | 2.92**; 5.96***; 2.92* |
| 13 | 2 | H | H | 2-(3-tert-butylphenoxy)phenyl | |
| 14 | 2 | H | H | 2-[4-chloro-3- | 2.58**; 4.9***; 2.58* |
| | | | | (trifluoromethyl)phenoxy]phenyl | |
| 15 | 2 | H | H | 2-(trifluoromethoxy)phenyl | 1.77**; 3.54***; 1.77* |
| 16 | 2 | acetyl | H | 2-(2,4-difluorophenoxy)phenyl | 4.36**; 4.32***; 4.36* |
| 17 | 2 | acetyl | H | 2-phenoxyphenyl | 4.41**; 4.39***; 4.41* |
| 18 | 2 | H | H | 3,4-difluorophenyl | 1.64**; 3.12***; 1.64* |
| 19 | 2 | H | H | 2,5-difluorophenyl | 1.58**; 3.16***; 1.58* |
| 20 | 2 | H | H | 2-chlorophenyl | 1.63**; 3.19***; 1.63* |
| 21 | 2 | H | H | 2-(trifluoromethyl)phenyl | 1.76**; 3.25***; 1.76* |
| 22 | 2 | H | H | 2-(prop-2-en-1-yloxy)phenyl | 1.83**; 3.45***; 1.83* |
| 23 | 2 | H | H | 2-ethoxyphenyl | 1.77**; 3.35***; 1.77* |
| 24 | 2 | H | H | 2,3-dichlorophenyl | 1.81**; 3.72***; 1.81* |
| 25 | 2 | H | H | 2,3-difluorophenyl | 1.58**; 3.21***; 1.58* |
| 26 | 2 | H | H | 2-fluoro-3-(trifluoromethyl)phenyl | 1.84**; 3.78***; 1.84* |
| 27 | 2 | H | H | 3,4-dimethoxyphenyl | 1.35**; 2.36***; 1.35* |
| 28 | 2 | H | H | 1,3-benzodioxol-5-yl | 1.41**; 2.65***; 1.41* |
| 29 | 2 | H | H | 3,5-difluorophenyl | 1.57**; 3.22***; 1.57* |
| 30 | 2 | H | H | 3-chloro-4-fluorophenyl | 1.76**; 3.45***; 1.76* |
| 31 | 2 | H | CH₃ | 4-chlorophenyl | 1.84**; 3.72***; 1.84* |
| 32 | 2 | H | H | 4-tert-butylphenyl | 2.2**; 4.35***; 2.2* |
| 33 | 2 | acetyl | H | 2-ethoxyphenyl | 2.71**; 5.03***; 2.71* |
| 34 | 2 | acetyl | H | 4-chlorophenyl | 3.78**; 3.78***; 3.78* |
| 35 | 2 | acetyl | H | 2,4-dichlorophenyl | 4.33**; 4.33***; 4.33* |
| 36 | 2 | acetyl | H | 2,3-dichlorophenyl | 4.03**; 4.02***; 4.03* |
| 37 | 2 | acetyl | H | 2,3-difluorophenyl | 3.63**; 3.63***; 3.63* |
| 38 | 2 | acetyl | H | 2-fluoro-3-(trifluoromethyl)phenyl | 4.25**; 4.23***; 4.25* |
| 39 | 2 | acetyl | H | 1,3-benzodioxol-5-yl | 2.96**; 2.95***; 2.96* |
| 40 | 2 | acetyl | H | 3,5-difluorophenyl | 3.63**; 3.62***; 3.63* |
| 41 | 2 | acetyl | H | 3-chloro-4-fluorophenyl | 3.88**; 3.88***; 3.88* |
| 42 | 2 | acetyl | CH₃ | 4-chlorophenyl | 3.92**; 3.96***; 3.92* |
| 43 | 2 | acetyl | CH₃ | 2,3-difluorophenyl | 3.37**; 3.37***; 3.37* |
| 44 | 2 | H | CH₃ | 2,3-difluorophenyl | 1.7**; 3.2***; 1.7* |
| 45 | 2 | H | H | 3-(trifluoromethyl)phenyl | 1.97**; 3.55*** |
| 46 | 2 | H | H | 3-bromophenyl | 1.8** |
| 47 | 2 | H | H | 3-(2,2-dimethylpropyl)phenyl | 2.51**; 4.86*** |
| 48 | 2 | H | H | 2-bromophenyl | 1.58**; 3.25*** |
| 49 | 2 | H | H | 4-(difluoromethoxy)phenyl | 1.44** |
| 50 | 2 | H | H | 4-(trifluoromethoxy)phenyl | 1.78** |
| 51 | 2 | H | H | 2,6-dichlorophenyl | 1.42** |
| 52 | 2 | H | H | 3,4-dichlorophenyl | 3.93***; 1.68* |
| 53 | 2 | H | H | 2-chloropyridin-3-yl | 0.71* |
| 54 | 2 | H | H | 4-fluorophenyl | 1.23* |
| 55 | 2 | H | H | 3,5-dichlorophenyl | 4.21***; 1.59* |
| 56 | 2 | H | H | 4-methoxyphenyl | 1.18* |
| 57 | 2 | H | H | 3-methoxyphenyl | 2.76***; 1.19* |
| 58 | 2 | H | H | 2,6-dimethylpyridin-3-yl | |
| 59 | 2 | H | H | 3-chlorophenyl | 1.37** |
| 60 | 2 | H | H | 3-methylphenyl | 1.32* |
| 61 | 2 | H | H | pyridin-2-yl | 0.27** |
| 62 | 2 | H | H | 6-methylpyridin-2-yl | 2.13***; 0.29* |
| 63 | 2 | H | H | 6-chloropyridin-3-yl | 2.38***; 0.94* |
| 64 | 2 | H | H | biphenyl-2-yl | 1.82** |
| 65 | 2 | H | H | 3′-methylbiphenyl-2-yl | 2** |
| 66 | 2 | H | H | 3′,4′-dichlorobiphenyl-2-yl | 2.2** |
| 67 | 2 | H | H | 4′-methoxybiphenyl-2-yl | 1.83** |
| 68 | 2 | H | H | 4′-methoxybiphenyl-3-yl | 3.88***; 1.85* |
| 69 | 2 | H | H | 2-chlorobenzyl | 1.39* |
| 70 | 2 | H | H | 3-chlorobenzyl | 3.36***; 1.58* |
| 71 | 2 | H | H | 3′-methylbiphenyl-3-yl | 4.48***; 2.12* |
| 72 | 2 | H | H | cyclopropylmethyl | 2.73***; 1.07* |
| 73 | 2 | H | H | 1-phenylethyl | 3.27***; 1.51* |
| 74 | 2 | H | H | 2-(4-chlorophenoxy)phenyl | 2.03* |
| 75 | 2 | H | H | 2-(4-fluorophenoxy)phenyl | 1.87* |
| 76 | 2 | H | H | 2-(2,6-difluorophenoxy)phenyl | 3.78***; 1.8* |
| 77 | 2 | H | H | 2-(2,3,4-trifluorophenoxy)phenyl | 4.17***; 2.01* |
| 78 | 2 | H | H | 2-(3,4-difluorophenoxy)phenyl | 4.18***; 1.83* |
| 79 | 2 | H | H | 2-(4-methylphenoxy)phenyl | 4.51***; 2.17* |
| 80 | 2 | H | H | 2-(2,5-difluorophenoxy)phenyl | 3.98***; 1.94* |
| 81 | 2 | H | H | 2-benzylphenyl | 2.02*** |
| 82 | 2 | H | H | 2-(2,4,6-trifluorophenoxy)phenyl | 3.96***; 2.02* |
| 83 | 2 | H | H | 2-(3-fluorophenoxy)phenyl | 1.95* |
| 84 | 2 | H | H | 2-(4-bromophenoxy)phenyl | 2.14* |
| 85 | 2 | H | H | 2-(3-chlorophenoxy)phenyl | 2.16* |
| 86 | 2 | H | H | 2-(2-bromophenoxy)phenyl | 4.38***; 2.06* |
| 87 | 2 | H | H | 2-(3-bromophenoxy)phenyl | 4.71***; 2.19* |
| 88 | 2 | H | H | 2-(2,4-difluorobenzyl)phenyl | 4.27***; 2.04* |
| 89 | 2 | H | H | 2-(3,5-dichlorobenzyl)phenyl | 2.43**; 5.35*** |
| 90 | 2 | H | H | 2-(4-fluorobenzyl)phenyl | 1.95**; 4.19*** |
| 91 | 2 | H | H | 2-(2,6-dichlorobenzyl)phenyl | 2.16**; 4.36*** |
| 92 | 2 | H | H | 2-(3,4-dichlorobenzyl)phenyl | 5.07***; 2.36* |
| 93 | 2 | H | H | 2-(4-chlorobenzyl)phenyl | 2.17**; 4.7*** |
| 94 | 2 | H | H | 4-benzylphenyl | 2.07**; 4.28*** |
| 95 | 1 | H | H | 2-(2,4-dimethylphenoxy)phenyl | 2.52**; 2.48*** |
| 96 | 1 | H | H | 4-chlorophenyl | 1.57** |
| 97 | 1 | H | H | 4-(2,4-difluorophenoxy)phenyl | 3.59***; 2.03* |
| 98 | 1 | H | H | 2-phenoxyphenyl | 2.05**; 3.54***; 1.92* |
| 99 | 1 | H | H | 2-(2,4-dichlorophenoxy)phenyl | 2.49**; 4.43***; 2.49* |
| 100 | 1 | H | H | 2-(3-tert-butylphenoxy)phenyl | |
| 101 | 1 | H | H | 2-(3-tert-butyl-4-chlorophenoxy)phenyl | 3.09**; 5.53***; 3.09* |
| 102 | 1 | H | H | 2-(2,4-difluorophenoxy)phenyl | 3.55***; 1.98* |
| 103 | 2 | H | H | 4-chlorophenyl | 1.68**; 3.47***; 1.68* |
| 104 | 2 | H | H | 2,4-dichlorophenyl | 1.76**; 3.92***; 1.76* |
| 105 | 2 | H | H | 3-(trifluoromethyl)phenyl | 1.75** |
| 106 | 2 | H | H | 3-bromophenyl | 1.53** |
| 107 | 2 | H | H | 2-bromophenyl | 1.38** |

The logP values were measured according to EEC directive 79/831 Annex V.A8 by HPLC (High Performance Liquid Chromatography) on reversed-phase columns (C 18), using the method below:
*The determination in the acidic range is carried out at pH 2.3 using the mobile phases 0.1% aqueous phosphoric acid and acetonitrile linear gradient from 10% acetonitrile to 95% acetonitrile.
**The LC-MS determination in the acidic range is carried out at pH 2.7 using the mobile phases 0.1% aqueous formic acid and acetonitrile (contains 0.1% formic acid) linear gradient from 10% acetonitrile to 95% acetonitrile
***The LC-MS determination in the neutral range is carried out at pH 7.8 using the mobile phases 0.001 molar aqueous ammonium bicarbonate solution and acetonitrile linear gradient from 10% acetonitrile to 95% acetonitrile.

Calibration was carried out using unbranched alkan-2-ones (having from 3 to 16 carbon atoms) with known logP values (the logP values were determined by the retention times using linear interpolation between two successive alkanones).

### Biological Examples

### Example 1

### Sphaerotheca Test (Cucumber)/Protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. 1 day after the treatment, the plants are inoculated with a spore suspension of Sphaerotheca fuliginea. The plants are then placed in a greenhouse at a relative atmospheric humidity of 70% and a temperature of 23° C.

Evaluation is carried out 7 days after the inoculation. 0% means an efficacy which corresponds to that of the control, whereas an efficacy of 100% means that no infection is observed.

In this test, the aminothiazoles according to the invention of the formulae 1, 2, 6, 7, 10, 11, 12, 13, 14, 15, 17, 19, 20, 21, 22, 23, 25, 26, 29, 30, 36, 37, 38, 40, 45, 46, 48, 52, 53, 55, 59, 60, 61, 67, 68, 69, 71, 73, 74, 75, 76, 77, 78, 79, 80, 81, 82, 83, 84, 85, 86, 87, 88, 89, 90, 91, 92, 93, 95, 98, 99, 102, 103, 104, 105, 106, 107 according to Table I show, at an active compound concentration of 500 ppm, an efficacy of 70% or more.

### Example 2

### Alternaria Test (Tomato)/Protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Alternaria solani. The plants are then placed in an incubation cabin at about 20° C. and 100% relative atmospheric humidity.

Evaluation is carried out 3 days after the inoculation. 0% means an efficacy which corresponds to that of the control, whereas an efficacy of 100% means that no infection is observed.

In this test, the aminothiazoles according to the invention of the formulae 7, 8, 17, 75, 81, 83, 86, 87, 88, 90, 93, 98 according to Table I show, at an active compound concentration of 100 ppm, an efficacy of 70% or more.

### Example 3

### Uromyces Test (Bean)/Protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the bean rust pathogen Uromyces appendiculatus and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the aminothiazoles according to the invention of the formulae 16, 17, 26, 75, 81, 83, 86, 90 according to Table I show, at an active compound concentration of 10 ppm, an efficacy of 70% or more.

### Example 4

### Puccinia triticina Test (Wheat)/Protective

| | |
|---|---|
| Solvent: | 49 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate.

After the spray coating has dried on, the plants are sprayed with spores with a spore suspension of Puccinia triticina. The plants remain in an incubation cabin at 20° C. and a relative atmospheric humidity of 100% for 48 hours.

The plants are placed in a greenhouse at a temperature of about 20° C. and a relative atmospheric humidity of about 80%.

Evaluation is carried out 8 days after the inoculation. 0% means an efficacy which corresponds to that of the control, whereas an efficacy of 100% means that no infection is observed.

In this test, the aminothiazoles according to the invention 7, 8, 9, 10, 11, 14, 16, 17, 22, 24, 26, 27, 68, 81, 82, 83, 84, 85, 87, 88, 90, 93, 98, 99, 102 according to Table I show, at an active compound concentration of 500 ppm, an efficacy of 70% or more.

1. A compounds of formula (I) 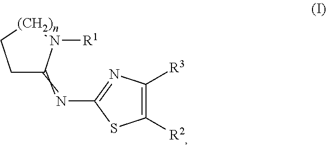
in which
R¹ is selected from the group consisting of hydrogen; straight-chain or branched C₁₋₁₂-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, —COOR′, —COR′, —CONR₂′, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN and —CONR₂′, where R′ is hydrogen or a straight-chain or branched C₁₋₁₂-alkyl, C₁₋₆-haloalkyl or cyclic C₃₋₈-alkyl group which may have 1 to 6 halogen atoms;
R² is selected from the group consisting of hydrogen and straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN and —CONR₂′;
R³ is selected from the group consisting of straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN, C₅₋₁₈-aryl, C₅₋₁₈-aryloxy, C₂₋₁₂-alkenyloxy, C₇₋₁₉-aralkyl and —CONR₂′, and where the C₅₋₁₈-aryl, C₅₋₁₈-aryloxy, C₂₋₁₂-alkenyloxy and C₇₋₁₉-aralkyl groups may be substituted by one, two or more radicals selected from the group consisting of —R′, —X, —OR′, —SR′, —NR′₂, —SiR′₃, —COOR′, —CN, aryloxy and —CONR₂′;
n is 0, 1 or 2;
and/or a salt, N-oxide, metal complex and/or stereoisomer thereof.

2. A compound of formula (I) according to claim 1, where
R¹ is selected from the group consisting of hydrogen, C₁₋₈-alkyl groups, —COOR′, —COR′, —CONR₂′;
R² is selected from the group consisting of hydrogen and straight-chain or branched C₁₋₈-alkyl groups;
R³ is selected from the group consisting of phenyl and phenyl-C₁₋₄-alkyl groups which may be substituted at the phenyl ring by one, two or more halogen atoms, phenyl, phenoxy, C₂₋₆-alkenyloxy, C₁₋₆-alkoxy, phenyl-C₁₋₄-alkyl groups or C₁₋₅-haloalkyl groups or which may be fused with the five-, six- or seven-membered carbocyclic or heterocyclic rings and which may have one or two heteroatoms selected from the group consisting of O and N in the ring, where all groups mentioned may optionally be substituted by halogen atoms, C₁₋₆-alkyl, C₁₋₆-alkoxy or C₁₋₅-haloalkyl groups;
n is 0 or 1;
and/or a salt, N-oxide, metal complex and/or stereoisomer thereof.

3. A compound of formula (I) according to claim 1, in which
R¹ is selected from the group consisting of hydrogen, C₁₋₈-alkyl groups, —COOR′, —COR′, —CONR₂′;
R² is hydrogen;
R³ is selected from
phenyl, benzyl or phenethyl groups which may be substituted by one, two or more halogen atoms, straight-chain or branched C₁₋₆-alkyl or C₁₋₆-haloalkyl groups or by phenyl or benzyl groups, where the latter phenyl or benzyl groups may be substituted by one, two or more halogen atoms, methyl or methoxy groups;
phenoxyphenyl or diphenoxyphenyl groups which may be substituted in the phenoxy radical by one or two halogen atoms, straight-chain or branched C₁₋₆-alkyl or C₁₋₆-haloalkyl groups;
C₂₋₆-alkenyloxyphenyl groups which may be substituted by one or two halogen atoms, straight-chain or branched C₁₋₆-alkyl or C₁₋₆-haloalkyl groups;
C₁₋₆-alkoxyphenyl groups which may be substituted by one or two halogen atoms, straight-chain or branched C₁₋₆-alkyl or C₁₋₆-haloalkyl groups;
1,3-benzodioxol-5-yl groups;
pyridyl groups which may be substituted by one or two halogen atoms, straight-chain or branched C₁₋₆-alkyl or C₁₋₆-haloalkyl groups;
C₃₋₆-cycloalkyl-C₁₋₄-alkyl groups;
n is 1;
and/or a salt, N-oxide, metal complex and/or a stereoisomer thereof.

4. A compound of formula (I) according to claim 1 selected from the group consisting of 4-[2-(2,4-dimethylphenoxy)phenyl]-N-[(2E/Z)-piperidin-2-ylidene]-1,3-thiazole-2-amine, 4-(4-chlorophenyl)-N-[(2E/Z)-piperidin-2-ylidene]-1,3-thiazole-2-amine, 4-(4-bromophenyl)-N-[(2E/Z)-piperidin-2-ylidene]-1,3-thiazole-2-amine, 4-(2,4-dichlorophenyl)-N-[(2E/Z)-piperidin-2-ylidene]-1,3-thiazole-2-amine, 4-(4-phenoxyphenyl)-N-[(2E/Z)-piperidin-2-ylidene]-1,3-thiazole-2-amine, 4-[3,5-bis(trifluoromethyl)phenyl]-N-[(2E/Z)-piperidin-2-ylidene]-1,3-thiazole-2-amine, 4-[2-(2,4-difluorophenoxy)phenyl]-N-[(2E/Z)-piperidin-2-ylidene]-1,3-thiazole-2-amine, 4-[2-chloro-3-(trifluoromethyl)phenyl]-N-[(2E/Z)-piperidin-2-ylidene]-1,3-thiazole-2-amine, 4-[4-(2,4-difluorophenoxy)phenyl]-N-[(2E/Z)-piperidin-2-ylidene]-1,3-thiazole-2-amine, 4-(2-phenoxyphenyl)-N-[(2E/Z)-piperidin-2-ylidene]-1,3-thiazole-2-amine, 4-[2-(2,4-dichlorophenoxy)phenyl]N-[(2E/Z)-piperidin-2-ylidene]-1,3-thiazole-2-amine, 4-[2-(3-tert-butyl-4-chlorophenoxy)phenyl]-N-[(2E/Z)-piperidin-2-ylidene]-1,3-thiazole-2-amine, 4-[2-(3-tert-butylphenoxy)phenyl]-N-[(2E/Z)-piperidin-2-ylidene]-1,3-thiazole-2-amine, 4-{2-[4-chloro-3-(trifluoromethyl)-phenoxy]phenyl}-N-[(2E/Z)-piperidin-2-ylidene]-1,3-thiazole-2-amine, N-[(2E/Z)-piperidin-2-ylidene]-4-[2-(trifluoromethoxy)phenyl]-1,3-thiazole-2-amine, 1-[(2E/Z)-2-({4-[2-(2,4-difluorophenoxy)phenyl]-1,3-thiazol-2-yl}imino)piperidin-1-yl]ethanone, 1-[(2E/Z)-2-{[4-(2-phenoxyphenyl)-1,3-thiazol-2-yl]imino}piperidin-1-yl]ethanone, 4-(3,4-difluorophenyl)-N-[(2E/Z)-piperidin-2-ylidene]-1,3-thiazole-2-amine, 4-(2,5-difluorophenyl)-N-[(2E/Z)-piperidin-2-ylidene]-1,3-thiazole-2-amine, 4-(2-chlorophenyl)-N-[(2E/Z)-piperidin-2-ylidene]-1,3-thiazole-2-amine, N-[(2E/Z)-piperidin-2-ylidene]-4-[2-(trifluoromethyl)phenyl]-1,3-thiazole-2-amine, N-[(2E/Z)-piperidin-2-ylidene]-4-[2-(prop-2-en-1-yloxy)phenyl]-1,3-thiazole-2-amine, 4-(2-ethoxyphenyl)-N-[(2E/Z)-piperidin-2-ylidene]-1,3-thiazole-2-amine, 4-(2,3-dichlorophenyl)-N-[(2E/Z)-piperidin-2-ylidene]-1,3-thiazole-2-amine, 4-(2,3-difluorophenyl)-N-[(2E/Z)-piperidin-2-ylidene]-1,3-thiazole-2-amine, 4-[2-fluoro-3-(trifluoromethyl)phenyl]-N-[(2E/Z)-piperidin-2-ylidene]-1,3-thiazole-2-amine, 4-(3,4-dimethoxyphenyl)-N-[(2E/Z)-piperidin-2-ylidene]-1,3-thiazole-2-amine, 4-(1,3-benzodioxol-5-yl)-N-[(2E/Z)-piperidin-2-ylidene]-1,3-thiazole-2-amine, 4-(3,5-difluorophenyl)-N-[(2E/Z)-piperidin-2-ylidene]-1,3-thiazole-2-amine, 4-(3-chloro-4-fluorophenyl)-N-[(2E/Z)-piperidin-2-ylidene]-1,3-thiazole-2-amine, 4-(4-chlorophenyl)-5-methyl-N-[(2E/Z)-piperidin-2-ylidene]-1,3-thiazole-2-amine, 4-(4-tert-butylphenyl)-N-[(2E/Z)-piperidin-2-ylidene]-1,3-thiazole-2-amine, 1-[(2E/Z)-2-{[4-(2-ethoxyphenyl)-1,3-thiazol-2-yl]imino}piperidin-1-yl]ethanone, 1-[(2E/Z)-2-{[4-(4-chlorophenyl)-1,3-thiazol-2-yl]imino}piperidin-1-yl]ethanone, 1-[(2E/Z)-2-{[4-(2,4-dichlorophenyl)-1,3-thiazol-2-yl]imino}piperidin-1-yl]ethanone, 1-[(2E/Z)-2-{[4-(2,3-dichlorophenyl)-1,3-thiazol-2-yl]imino}piperidin-1-yl]ethanone, 1-[(2E/Z)-2-{[4-(2,3-difluorophenyl)-1,3-thiazol-2-yl]imino}piperidin-1-yl]ethanone, 1-[(2E/Z)-2-({-4-[2-fluoro-3-(trifluoromethyl)phenyl]-1,3-thiazol-2-yl}imino)piperidin-1-yl]ethanone, 1-[(2E/Z)-2-{[4-(1,3-benzodioxol-5-yl)-1,3-thiazol-2-yl]imino}piperidin-1-yl]ethanone, 1-[(2E/Z)-2-{[4-(3,5-difluorophenyl)-1,3-thiazol-2-yl]imino}piperidin-1-yl]ethanone, 1-[(2E/Z)-2-{[4-(3-chloro-4-fluorophenyl)-1,3-thiazol-2-yl]imino}piperidin-1-yl]ethanone, 1-[(2E/Z)-2-{[4-(4-chlorophenyl)-5-methyl-1,3-thiazol-2-yl]imino}piperidin-1-yl]ethanone, 1-[(2E/Z)-2-{[4-(2,3-difluorophenyl)-5-methyl-1,3-thiazol-2-yl]imino}piperidin-1-yl]ethanone, 4-(2,3-difluorophenyl)-5-methyl-N-[(2E/Z)-piperidin-2-ylidene]-1,3-thiazole-2-amine, N-[(2E/Z)-piperidin-2-ylidene]-4-[3-(trifluoromethyl)phenyl]-1,3-thiazole-2-amine, 4-(3-bromophenyl)-N-[(2E/Z)-piperidin-2-ylidene]-1,3-thiazole-2-amine, 4-[3-(2,2-dimethylpropyl)phenyl]-N-[(2E/Z)-piperidin-2-ylidene]-1,3-thiazole-2-amine, 4-(2-bromophenyl)-N-[(2E/Z)-piperidin-2-ylidene]-1,3-thiazole-2-amine, 4-[4-(difluoromethoxy)phenyl]-N-[(2E/Z)-piperidin-2-ylidene]-1,3-thiazole-2-amine, N-[(2E/Z)-piperidin-2-ylidene]-4-[4-(trifluoromethoxy)phenyl]-1,3-thiazole-2-amine, 4-(2,6-dichlorophenyl)-N-[(2E/Z)-piperidin-2-ylidene]-1,3-thiazole-2-amine, 4-(3,4-dichlorophenyl)-N-[(2E/Z)-piperidin-2-ylidene]-1,3-thiazole-2-amine, 4-(2-chloropyridin-3-yl)-N-[(2E/Z)-piperidin-2-ylidene]-1,3-thiazole-2-amine, 4-(4-fluorophenyl)-N-[(2E/Z)-piperidin-2-ylidene]-1,3-thiazole-2-amine, 4-(3,5-dichlorophenyl)-N-[(2E/Z)-piperidin-2-ylidene]-1,3-thiazole-2-amine, 4-(4-methoxyphenyl)-N-[(2E/Z)-piperidin-2-ylidene]-1,3-thiazole-2-amine, 4-(3-methoxyphenyl)-N-[(2E/Z)-piperidin-2-ylidene]-1,3-thiazole-2-amine, 4-(2,6-dimethylpyridin-3-yl)-N-[(2E/Z)-piperidin-2-ylidene]-1,3-thiazole-2-amine, 4-(3-chlorophenyl)-N-[(2E/Z)-piperidin-2-ylidene]-1,3-thiazole-2-amine, 4-(3-methylphenyl)-N-[(2E/Z)-piperidin-2-ylidene]-1,3-thiazole-2-amine, N-[(2E/Z)-piperidin-2-ylidene]-4-(pyridin-2-yl)-1,3-thiazole-2-amine, 4-(6-methylpyridin-2-yl)-N-[(2E/Z)-piperidin-2-ylidene]-1,3-thiazole-2-amine, 4-(6-chloropyridin-3-yl)-N-[(2E/Z)-piperidin-2-ylidene]-1,3-thiazole-2-amine, 4-(biphenyl-2-yl)-N-[(2E/Z)-piperidin-2-ylidene]-1,3-thiazole-2-amine, 4-(3′-methylbiphenyl-2-yl)-N-[(2E/Z)-piperidin-2-ylidene]-1,3-thiazole-2-amine, 4-(3′,4′-dichlorobiphenyl-2-yl)-N-[(2E/Z)-piperidin-2-ylidene]-1,3-thiazole-2-amine, 4-(4′-methoxybiphenyl-2-yl)-N-[(2E/Z)-piperidin-2-ylidene]-1,3-thiazole-2-amine, 4-(4′-methoxybiphenyl-3-yl)-N-[(2E/Z)-piperidin-2-ylidene]-1,3-thiazole-2-amine, 4-(2-chlorobenzyl)-N-[(2E/Z)-piperidin-2-ylidene]-1,3-thiazole-2-amine, 4-(3-chlorobenzyl)-N-[(2E/Z)-piperidin-2-ylidene]-1,3-thiazole-2-amine, 4-(3′-methylbiphenyl-3-yl)-N-[(2E/Z)-piperidin-2-ylidene]-1,3-thiazole-2-amine, 4-(cyclopropylmethyl)-N-[(2E/Z)-piperidin-2-ylidene]-1,3-thiazole-2-amine, 4-(1-phenylethyl)-N-[(2E/Z)-piperidin-2-ylidene]-1,3-thiazole-2-amine, 4-[2-(4-chlorophenoxy)phenyl]-N-[(2E/Z)-piperidin-2-ylidene]-1,3-thiazole-2-amine, 4-[2-(4-fluorophenoxy)phenyl]-N-[(2E/Z)-piperidin-2-ylidene]-1,3-thiazole-2-amine, 4-[2-(2,6-difluorophenoxy)phenyl]-N-[(2E/Z)-piperidin-2-ylidene]-1,3-thiazole-2-amine, N-[(2E/Z)-piperidin-2-ylidene]-4-[2-(2,3,4-trifluorophenoxy)phenyl]-1,3-thiazole-2-amine, 4-[2-(3,4-difluorophenoxy)phenyl]-N-[(2E/Z)-piperidin-2-ylidene]-1,3-thiazole-2-amine, 4-[2-(4-methylphenoxy)phenyl]-N-[(2E/Z)-piperidin-2-ylidene]-1,3-thiazole-2-amine, 4-[2-(2,5-difluorophenoxy)phenyl]-N-[(2E/Z)-piperidin-2-ylidene]-1,3-thiazole-2-amine, 4-(2-benzylphenyl)-N-[(2E/Z)-piperidin-2-ylidene]-1,3-thiazole-2-amine, N-[(2E/Z)-piperidin-2-ylidene]-4-[2-(2,4,6-trifluorophenoxy)phenyl]-1,3-thiazole-2-amine, 4-[2-(3-fluorophenoxy)phenyl]-N-[(2E/Z)-piperidin-2-ylidene]-1,3-thiazole-2-amine, 4-[2-(4-bromophenoxy)phenyl]-N-[(2E/Z)-piperidin-2-ylidene]-1,3-thiazole-2-amine, 4-[2-(3-chlorophenoxy)phenyl]-N-[(2E/Z)-piperidin-2-ylidene]-1,3-thiazole-2-amine, 4-[2-(2-bromophenoxy)phenyl]-N-[(2E/Z)-piperidin-2-ylidene]-1,3-thiazole-2-amine, 4-[2-(3-bromophenoxy)phenyl]-N-[(2E/Z)-piperidin-2-ylidene]-1,3-thiazole-2-amine, 4-[2-(2,4-difluorobenzyl)phenyl]-N-[(2E/Z)-piperidin-2-ylidene]-1,3-thiazole-2-amine, 4-[2-(3,5-dichlorobenzyl)phenyl]-N-[(2E/Z)-piperidin-2-ylidene]-1,3-thiazole-2-amine, 4-[2-(4-fluorobenzyl)phenyl]-N-[(2E/Z)-piperidin-2-ylidene]-1,3-thiazole-2-amine, 4-[2-(2,6-dichlorobenzyl)phenyl]-N-[(2E/Z)-piperidin-2-ylidene]-1,3-thiazole-2-amine, 4-[2-(3,4-di-chlorobenzyl)phenyl]-N-[(2E/Z)-piperidin-2-ylidene]-1,3-thiazole-2-amine, 4-[2-(4-chloro-benzyl)phenyl]-N-[(2E/Z)-piperidin-2-ylidene]-1,3-thiazole-2-amine, 4-(4-benzylphenyl)-N-[(2E/Z)-piperidin-2-ylidene]-1,3-thiazole-2-amine, 4-[2-(2,4-dimethylphenoxy)phenyl]-N-[(2E/Z)-pyrrolidin-2-ylidene]-1,3-thiazole-2-amine, 4-(4-chlorophenyl)-N-[(2E/Z)-pyrrolidin-2-ylidene]-1,3-thiazole-2-amine, 4-[4-(2,4-difluorophenoxy)phenyl]-N-[(2E/Z)-pyrrolidin-2-ylidene]-1,3-thiazole-2-amine, 4-(2-phenoxyphenyl)-N-[(2E/Z)-pyrrolidin-2-ylidene]-1,3-thiazole-2-amine, 4-[2-(2,4-dichlorophenoxy)phenyl]-N-[(2E/Z)-pyrrolidin-2-ylidene]-1,3-thiazole-2-amine, 4-[2-(3-tert-butylphenoxy)phenyl]-N-[(2E/Z)-pyrrolidin-2-ylidene]-1,3-thiazole-2-amine, 4-[2-(3-tert-butyl-4-chlorophenoxy)phenyl]-N-[(2E/Z)-pyrrolidin-2-ylidene]-1,3-thiazole-2-amine, 4-[2-(2,4-difluorophenoxy)phenyl]-N-[(2E/Z)-pyrrolidin-2-ylidene]-1,3-thiazole-2-amine, 4-(4-chlorophenyl)-N-[(2E/Z)-piperidin-2-ylidene]-1,3-thiazole-2-amine, 4-(2,4-dichlorophenyl)-N-[(2E/Z)-piperidin-2-ylidene]-1,3-thiazole-2-amine, N-[(2E/Z)-piperidin-2-ylidene]-4-[3-(trifluoromethyl)phenyl]-1,3-thiazole-2-amine, 4-(3-bromophenyl)-N-[(2E/Z)-piperidin-2-ylidene]-1,3-thiazole-2-amine, and 4-(2-bromophenyl)-N-[(2E/Z)-piperidin-2-ylidene]-1,3-thiazole-2-amine.

5. A process for preparing a compound of formula (I) according to claim 1, which comprises at least one of (c) or (d) below:
(c) reacting a 2-aminothiazole of the formula (IV) with a lactam of the formula (V), in the presence of a condensing agent, to give a compound of formula (Ia) 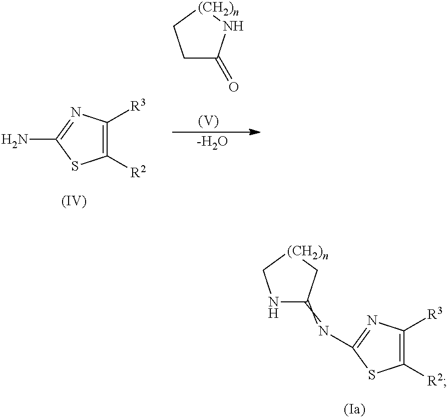
(d) reacting a compound of the formula (Ia) with a carboxylic anhydride of the formula (VI) to give a compound of formula (Ib)

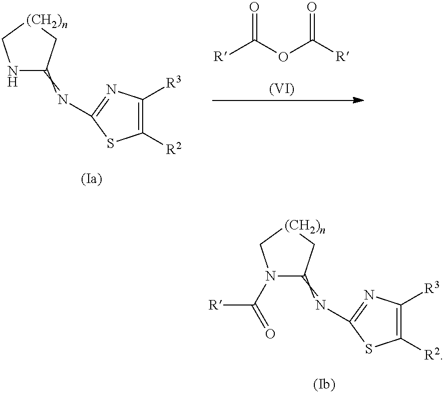

6. A compound of formula (IV) 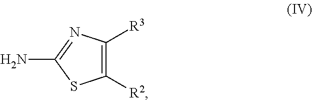
in which
R² is selected from the group consisting of hydrogen and straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN and —CONR₂′;
R³ is selected from the group consisting of straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN, C₅₋₁₈-aryl, C₅₋₁₈-aryloxy, C₂₋₁₂-alkenyloxy, C₇₋₁₉-aralkyl and —CONR₂′, and where the C₅₋₁₈-aryl, C₅₋₁₈-aryloxy, C₂₋₁₂-alkenyloxy and C₇₋₁₉-aralkyl groups may be substituted by one, two or more radicals selected from the group consisting of —R′, —X, —OR′, —SR′, —NR′₂, —SiR′₃, —COOR′, —CN, aryloxy and —CONR₂′;
n is 0, 1 or 2;
and/or a salt, N-oxide, metal complex and/or stereoisomer thereof.

7. A compound of formula (Ia) 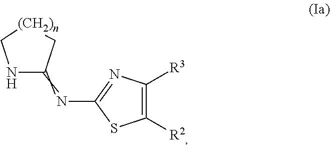
in which
R² is selected from the group consisting of hydrogen and straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN and —CONR₂′;
R³ is selected from the group consisting of straight-chain or branched C₁₋₆-alkyl, C₂₋₁₂-alkenyl, C₂₋₁₂-alkynyl, cyclic C₃₋₈-alkyl, C₄₋₈-alkenyl, C₄₋₈-alkynyl or C₅₋₁₈-aryl, C₇₋₁₉-aralkyl and C₇₋₁₉-alkaryl groups, where, in a ring system of all cyclic groups mentioned above, one or more carbon atoms may optionally be replaced by one or more heteroatoms selected from the group consisting of N, O, P and S, and all groups mentioned above may be substituted by one or more groups selected from the group consisting of —R′, —X, —OR′, —SR′, —NR′₂, —SiR′₃, —COOR′, —CN, C₅₋₁₈-aryl, C₅₋₁₈-aryloxy, C₂₋₁₂-alkenyloxy, C₇₋₁₉-aralkyl and —CONR₂′, and where the C₅₋₁₈-aryl, C₅₋₁₈-aryloxy, C₂₋₁₂-alkenyloxy and C₇₋₁₉-aralkyl groups may be substituted by one, two or more radicals selected from the group consisting of —R′, —X, —OR′, —SR′, —NR′₂, —SiR′₃, —COOR′, —CN, aryloxy and —CONR₂′;
n is 0, 1 or 2;
and/or a salt, N-oxide, metal complex and/or stereoisomer thereof.

8. A composition for controlling unwanted microorganisms, comprising at least one compound according to claim 1.

9. A compound of according to claim 1 and/or a mixture thereof which is capable of controlling unwanted microorganisms.

10. A method for controlling unwanted microorganisms, comprising applying a compound according to claim 1 to the microorganisms and/or a habitat thereof.

11. Seed which is treated with at least one compound according to claim 1.

12. Seed treated with at least one compound of claim 2.

13. Seed treated with at least one compound of claim 3.

14. Seed treated with at least one compound of claim 4.

15. A compound which has been produced through a starting material and/or an intermediate comprising a compound of claim 7.

16. A compound which has been produced through a starting material and/or an intermediate comprising a compound of claim 6.

17. A composition for controlling unwanted microorganisms, comprising at least one compound according to claim 4.

18. A compound of according to claim 4 and/or a mixture thereof which is capable of controlling unwanted microorganisms.

19. A method for controlling unwanted microorganisms, comprising applying a compound according to claim 4 to the microorganisms and/or a habitat thereof.
